(19) United States
(12) Patent Application Publication
(10) Pub. No.: US 2021/0261957 A1
(43) Pub. Date: Aug. 26, 2021

(54) VESICLES FOR TRACELESS DELIVERY OF GUIDE RNA MOLECULES AND/OR GUIDE RNA MOLECULE/RNA-GUIDED NUCLEASE COMPLEX(ES) AND A PRODUCTION METHOD THEREOF

(71) Applicant: ALIA THERAPEUTICS S.R.L., Trento (IT)

(72) Inventors: Gianluca PETRIS, Udine (IT); Antonio CASINI, Trento (IT); Anna CERESETO, Trento (IT)

(21) Appl. No.: 17/259,165

(22) PCT Filed: Jul. 8, 2019
(86) PCT No.: PCT/IB2019/055805

§ 371 Date: Jan. 9, 2021

(30) Foreign Application Priority Data
Jul. 10, 2018 (IT) 102018000007055

(51) Int. Cl. C12N 15/11; C12N 9/22; C12N 15/88

(52) U.S. Cl. CPC C12N 15/11; C12N 9/22; C12N 2800/80; C12N 2320/32; C12N 2310/20; C12N 15/88

(57) ABSTRACT

The present invention describes vesicles carrying gRNA(s) and/or CRISPR-associated RNA guided-nuclease ribonucleoprotein complexes (RNPs) that are efficiently delivered into target cells through fusogenic envelope proteins.

### FIELD OF THE INVENTION

The present invention refers to engineered vesicles for direct guide RNA molecules and/or guide RNA molecule/RNA-guided nuclease complex(es) delivery into a target cell as well as a production method thereof.

### BACKGROUND ART

The direct delivery of guide RNA(s) (gRNAs) and/or RNA-guide nuclease ribonucleoprotein complex(es) (RNP(s)) into target cells can find several applications in biotechnology, cell research, diagnostics and therapy. This process consists in the introduction into the cell cytoplasm and/or nucleus of desired gRNAs and/or RNPs molecules through chemical or physical methods, while reducing the cellular toxicity to a minimum. Current methods include: transfection through lipid-based reagents, polymer-based reagents or protein-based reagents; electroporation; microinjection; fusion to cell penetrating peptides; introduction through viral-like particles (VLPs). While all these methods are diffused in research practice, their efficacy and toxicity profile varies according to the cell line model used in the experiments. Moreover, the majority of these techniques are not suitable for the in vivo delivery of RNAs or RNPs in animal models or for therapeutic purposes due to the low efficiency in cargo transfer. For example, electroporation is known to cause high levels of cellular toxicity and lacks a precise control of the amount of delivered RNPs or RNAs, while VLP-mediated delivery may be connected with enhanced immune responses against the delivery vehicle due to the presence of viral proteins used for packaging.

On the same line, obtaining considerable amounts of recombinant proteins to be used for direct delivery into cells can be challenging. Some examples of problems encountered during recombinant protein production can be: yield of the preparations, solubility of the recombinant protein, faithful recapitulation of post-translational modifications, faithful recapitulation of the folding of the original molecule.

Another major obstacle for the delivery of gRNAs and RNPs into cells is represented by the fact that standard methods for RNA expression are not suitable or show poor efficiency in the incorporation of certain types of transcripts into cytoplasm-derived vesicles. Moreover, several RNA post-transcriptional modifications can be detrimental or required on the gRNA to be delivered. As a consequence, the same difficulties are present when packaging RNPs containing those types of transcripts in such vesicles. In particular, RNAs without a 5′-cap, a 3′-poly-A tail or RNAs that have not been spliced or are not processed by RNA interference miRNA processing machinery that are transcribed in the nucleus do not reach the cytoplasm efficiently. Transcripts obtained from RNA polymerase III promoters (e.g. U6 promoter, H1 promoter, tRNA promoters, etc.), which are commonly used for biotechnological applications, fall in this category and are thus poorly packaged into cytoplasm-derived vesicles. RNA polymerase II transcript are generally exported to the cytoplasm for translation but are modified by 5′capping, intron splicing, base editing and 3′poly-A tailing before being exported from the nucleus, but such modifications may be undesirable for several applications and these RNAs are frequently trapped in cytosolic sub-compartments (e.g. RNA bodies) or in the cellular translational machinery (e.g. ribosomes).

The expression of uncapped and non-polyadenylated transcripts into the cytoplasm can be obtained by expressing an ectopic RNA polymerase in the cytoplasm of a desired cell. A corresponding promoter needs to be added upstream of the target transcript, while termination of transcription can be achieved by the presence of a suitable terminator sequence or by run-off transcription of a linear DNA template. The transcribed RNAs are often self-processed to remove part of the transcript (e.g. terminator sequences) by the presence of ribozymes (e.g. HDV and HH ribozymes). An example of such polymerase is the T7 RNA polymerase obtained from the T7 bacteriophage in combination with the T7 promoter.

Usually T7 RNA polymerase transcription has been used for in vitro cell free transcription of RNAs which are often combined with proteins for RNPs formation before their direct delivery into target cells or in in vitro assays.

Among the different applications exploiting direct delivery of RNPs into cells, one of particular interest is CRISPR-nuclease-mediated genome editing. CRISPR associated nucleases, generally called CRISPR-Cas (e.g. CRISPR-Cas9, CRISPR-Cpf1, CRISPR-Cas13), are families of nucleases (the most famous is Streptococcus pyrogenes Cas9, SpCas9) which in their natural form use one or more RNA molecules, forming a guide-RNA (gRNA), to recognize their target nucleic acid. The CRISPR-nuclease technologies have tremendous potential both for basic and clinical applications (Cong et al. 2013; Casini et al. 2018). The outcome of genome editing mediated by CRISPR-Cas9 highly depends on the efficiency of RNA-guided nuclease delivery in target cells and its specificity (extent of off-target activity). Since Cas9 non-specific cleavages correlate with high protein levels and long-term expression in recipient cells (Tsai & Joung 2016; Petris et al. 2017), delivery strategies are of crucial importance for both efficiency and specificity of Cas9 genome editing. Consistently, since the transient nature of the RNPs in target cells reduces unspecific cleavages at off-target sites, genome editing is preferably performed through the delivery of Cas9-gRNA ribonucleoprotein complexes (RNPs) (Ramakrishna et al. 2014; Zuris et al. 2015; S. Kim et al. 2014). Opposed to these methods, viral vectors, including those of retroviral origin, are widely used for efficient delivery of nuclease and gRNA genes both in vitro and in vivo (Long et al. 2016; Yang et al. 2016; Diao et al. 2016). Nevertheless, these delivery tools are generally not ideal for transient therapeutic approaches, due to long-term transgene expression and potential risks for insertional mutagenesis (Petris et al. 2017; Chick et al. 2012). Non-integrating viral vectors, such as those derived from adeno-associated viruses (AAV), are efficient for gene delivery and in principle should prevent mutagenic integration (Nakai et al. 2001; Lombardo et al. 2007; Zacchigna et al. 2014; Ruozi et al. 2015). However, these vectors are more suitable for long-term expression of small transgenes (not greater than ˜4 kb) and are thus not fully compatible with the CRISPR-nuclease technology, in particular with the most used SpCas9 and AsCpf1 nucleases. To circumvent the genotoxicity generated by retroviral vectors while preserving the viral delivery efficiency, a non-integrating lentiviral (IDLV) vector carrying the gRNA expression cassette packaged with the SpCas9 protein has been developed (Choi et al. 2016). A major improvement towards complete traceless delivery of exogenous protein cargos into cells is potentially offered by the development of viral-like particles (VLPs) (Voelkel et al. 2010). The viral origin of VLPs assures the efficient transduction of target cells, even though no viral genomic DNA is carried by the particles, thus allowing the rapid clearance of the shuttled protein and RNAs. VLPs have been mainly used in the past decade for vaccination purposes (Ramqvist et al. 2007), or for the delivery of exogenous proteins (Voelkel et al. 2010). To minimize the viral elements, protein cargo delivery can be obtained with vesicles made exclusively with the envelope glycoprotein of the vesicular stomatitis virus (VSV-G) (Mangeot et al. 2011).

### SUMMARY OF THE INVENTION

The object of this disclosure is to provide novel systems able to deliver in a transient way gRNA(s) and/or gRNA(s)/RNA-guided nuclease complexes into a target cell.

According to the invention, the above object is achieved thanks to the subject matter recalled specifically in the ensuing claims, which are understood as forming an integral part of this disclosure.

The present invention discloses a vesicle which is loaded with gRNAs or gRNAs complexed with a nuclease and a method to produce said vesicle. The gRNAs are accumulated into the cell cytoplasm using a cytoplasmic transcription system to obtain highly efficient incorporation of the gRNA into the vesicle. Such cytoplasmic transcription system can be operated exclusively in permissive cells.

The present invention provides a vesicle comprising:

i) a lipid envelope associated with at least one membrane-associated protein;

ii) at least one guide RNA molecule; and

iii) optionally at least one RNA-guided nuclease;

wherein the vesicle has at least one of the following features:
the at least one guide RNA molecule is present within the vesicle in an amount of at least 0.2% w/w to total vesicle protein mass; and
if the vesicle contains also the at least one RNA-guided nuclease, the at least one guide RNA molecule is complexed to the at least one RNA-guided nuclease, wherein the at least one guide RNA molecule is present within the vesicle in a molar ratio with respect to the at least one RNA-guided nuclease equal or above to 0.85:1.

Described herein is a new method that allows efficient packaging of gRNA molecules and/or nuclease ribonucleoprotein complexes (RNPs) within cytoplasm-derived structures released from a cell, i.e. vesicles.

According to the instant invention, the vesicle is produced by a method comprising the following steps:

i) providing a packaging cell, wherein the packaging cell has the following features:
a) the cell is tolerant to high levels of direct cytosolic transcription, wherein the cell tolerance is determined by: a lack of expression in the cell of at least one RNA virus-sensing pathway selected from: RIG-I, RIG-I-like protein, DDX58, MDA-5, IPS-1, RIPI, FADD, TRAF6, TRAF3, TANK, NAP, NEMO, IKKα, IKKβ, IKKε, IKKγ, TBK1, DDX3, IκB, NF-κB, IRF3, IRF7, p65, p50, RIP1, TLR3, TLR7, TLR8, INF-α, INF-β, INF-γ1, INF-γ2, INF-γ3, Caspase-1; and/or a cell ability to cap at least one guide-RNA molecule by expression of capping enzymes; and/or a cell ability to express a 5′ phosphatase for de-phosphorylating 5′triphosphate transcripts; and
b) the cell stably or transiently expresses an RNA polymerase in the cytoplasm;

ii) transfecting the packaging cell with at least one first expression cassette, and at least one second expression cassette and optionally at least one third expression cassette, wherein:
a) the first expression cassette comprises a first nucleotide sequence to transcribe at least one guide RNA molecule;
b) the second expression cassette comprises a second nucleotide sequence encoding at least one membrane-associated protein; and
c) the third expression cassette comprises a third nucleotide sequence encoding at least one RNA-guided nuclease;

iii) producing the vesicle from the packaging cell.

### BRIEF DESCRIPTION OF THE DRAWINGS

The invention will now be described in detail, purely by way of illustrative and non-limiting example, with reference to the attached figures, wherein:

FIG. 1. Design and genome editing activity of VEsiCas.

(a) EGFP disruption assay with VSV-G/SpCas9 vesicles produced in HEK293T cells. Percentages of EGFP knockout HEK293-EGFP cells generated by transfection of SpCas9 (SpCas9 plasmid) together with EGFP targeting (sgEGFP5) or control (sgCtr) sgRNA, or transduction with VSV-G/SpCas9 vesicles carrying U6 transcribed sgRNA. Where indicated HEK293-EGFP cells were pre-transfected with sgEGFP5 or sgCtr (+pre-sgRNA) prior to VSV-G/SpCas9 vesicles treatment. Data presented as mean±s.e.m. for n=2 independent experiments. (b) Scheme of VEsiCas production from BSR-T7/5 cells. T7 RNA polymerase, expressed in the cytosol, regulates the cytosolic sgRNA expression by mean of the T7 promoter. Vesicles decorated with VSV-G, expressed by the BSR-T7/5 producer cells, bud incorporating SpCas9 complexed with sgRNA to form VEsiCas. In target cells, VEsiCas release active SpCas9-sgRNA complexes, that enter the nuclei through two nuclear localization sequences introduced in SpCas9. (c) Genome activity of VEsiCas produced in BSR-T7/5 on HEK293-EGFP cells. Percentages of non-fluorescent HEK293-EGFP cells following transfection of SpCas9 (SpCas9 plasmid) together with sgRNAs (sgEGFP5 or sgCtr) or treatments with VEsiCas carrying sgRNAs (sgEGFP5 or sgCtr) either with or without pre-transfection with sgRNAs as indicated. Data presented as mean±s.e.m. for n=2 independent experiments. (d,e) VEsiCas-mediated editing of the CXCR4 (d) and VEGFA site3 (e) genomic loci. Percentages of indels formation in HEK293T cells measured through TIDE analysis following transfection of SpCas9 (Cas9 plasmid) together with sgRNAs (sgCXCR4, sgVEGFA site3 or sgCtr) or after three sequential treatments with VEsiCas carrying sgRNAs (sgCXCR4, sgVEGFA site3 or sgCtr). Data presented as mean±s.e.m. for n=2 independent experiments.

FIG. 2. Development of VSV-G vesicles for SpCas9 delivery.

(a) Cas9/VSV-G vesicles production and delivery in HEK293T cells. Western blot analysis of SpCas9 expression in cell extracts of producing cells (left panel), in the supernatant of producing cells (middle panel) and in target cells 6 hours post transduction (right panel). Ctr corresponds to cells transfected with an empty control plasmid, SpCas9 corresponds to cells over-expressing SpCas9 and sgRNA (sgEGFP5), SpCas9/VSV-G corresponds to cells over-expressing SpCas9, sgEGFP5 and VSV-G. Western blot is representative of n=2 independent experiments. (b) EGFP disruption assay in different cell lines using a U6 or T7 promoter sgRNA expression systems. Fluorescence microscopy images obtained from HEK293T, BHK21, BSR-T7/5 (a BHK21 clone stably expressing the T7 RNA polymerase) and Vero cell lines transfected with EGFP and SpCas9 expression plasmids together with plasmids expressing either EGFP-targeting (sgEGFP5) or non-targeting (sgCtr) sgRNAs from a U6 or a T7 promoter, as indicated. All cells but BSR-T7/5 were also co-transfected with a plasmid expressing the T7 RNA polymerase. EGFP knock-out was detected with variable intensity in all cell lines expressing sgRNAs (sgEGFP5) driven by the U6 promoter (right panels). Conversely, the sgRNA driven by the T7 RNA Polymerase system was able to induce EGFP knock-out only in permissive cells (BHK-21, BSR-T7/5 and Vero cells) but not in HEK293T cells. Scale bar: 100 μm. Data are representative of n=2 independent experiments. (c) Western blot analysis of SpCas9 detected in the supernatant of BSR-T7/5 (VEsiCas) or HEK293T producing cells (SpCas9/VSV-G). The gel was loaded with similar amounts of SpCas9 protein. Western blots were developed with anti-SpCas9 or anti-tubulin antibodies. Western blot is representative of n=2 independent experiments.

FIG. 3. Lentiviral-based viral-like particles (lenti-VLPs) for SpCas9 delivery.

(a) Scheme of the Gag-SpCas9 (Gag-Cas9) and MinimalGag-SpCas9 (MinGag-Cas9) chimeras. The domains of Gag, Matrix (MA), Capsid (CA), Nucleocapsid (NC) and peptides p1, p2 and p6, are indicated. A linker peptide separates Gag from SpCas9. The position of the nuclear localization signals (NLS) and the 3×FLAG-tag are indicated. MinGag-Cas9 fusion includes the N-terminal myristylation signal of MA, the C-terminal part of CA and the p2 peptide. The NC was substituted with the GCN4 leucine zipper domain (Z) to maintain particle assembly. The RSV p2b peptide substitutes p6 for particle formation (Accola et al. 2000). (b) Western blot analysis of Gag-SpCas9 and MinGag-SpCas9. Cells were transfected with plasmid encoding Gag-SpCas9 and MinimalGag-SpCas9 (MinGag-SpCas9) either containing (+Met) or not (−Met) a methionine between the FLAG and the linker peptide. The arrowhead indicates free SpCas9 probably generated by translation starting from the internal Met. Ctr corresponds to cells transfected with an empty control plasmid. Western blot is representative of n=2 independent experiments. (c) Activity of Gag-SpCas9 and MinGag-SpCas9 chimeras in EGFP disruption assay. HEK293-EGFP cells were transfected with plasmids expressing Gag-SpCas9 or MinGag-SpCas9 with or without the methionine between the FLAG and the linker peptide. Cells were also co-transfected with sgEGFP5 or sgCtr. NT=not treated. Data presented as mean±s.e.m. for n=2 independent experiments. (d) Western blot analysis of producing cells (Cells) and derived supernatants (VLPs) after overexpression of SpCas9, Gag-SpCas9 or MinGag-SpCas9 as indicated. Ctr corresponds to cells transfected with an empty control plasmid. Westernblot is representative of n=2 independent experiments. (e-g) Genome editing with lenti-VLPs. Editing activity induced by VSV-G-decorated Gag-SpCas9 or MinGag-SpCas9 lenti-VLPs towards (e) the EGFP (percentage of EGFP negative cells), (f) the CXCR4 or (g) the VEGFA site3 loci in HEK293T cells. The percentage of indels was analyzed by TIDE). Data presented as mean±s.e.m. for n=2 independent experiments.

FIG. 4. Genome editing by Multiplexing VEsiCas (a) Gene deletion using VEsiCas. Gel electrophoresis analysis of the EGFP locus deletion obtained in HEK293-EGFP cells through Multi-VEsiCas (left panel) treatment or by transfection with SpCas9 together with specific sgRNAs (sgEGFP5 and sgEGFPBi) (right panel). The amount of deletion (%) was quantified by densitometry. Arrowheads indicate the expected band corresponding to PCR amplification of the deleted EGFP locus. (b) Activity of VEsiCas delivering SpCas9 nickase. Percentages of EGFP knock-out cells following incubation with VEsiCas-n, carrying SpCas9 nickase loaded with sgRNA towards two sites in the EGFP locus (sgEGFP3gW and sgEGFPBi) or with sgCtr. NT=not treated. Data presented as mean±s.e.m. for n=2 independent experiments.

FIG. 5. Titration of VEsiCas and comparison with a widely used Cas9-sgRNA RNPs delivery method.

EGFP disruption assay in HEK293-EGFP cells treated with scalar amounts of SpCas9 delivered through VEsiCas or RNPs electroporation. Both RNPs and VEsiCas were loaded with the same sgRNA (sgEGFP5) transcribed by T7 RNA Polymerase either in vitro or in BSR-T7/5 cells respectively. Data are representative of n=2 independent experiments.

FIG. 6. VEsiCas mediated EGFP knock-out in J-Lat-A1 and HeLa cells.

J-Lat-A1 and HeLa stably expressing EGFP were treated with VEsiCas carrying EGFP targeting (sgEGFP5) or control (sgCtr) sgRNAs. The graph reports the percentages of non-fluorescent cells seven days following treatment. Data are representative of n=2 independent experiments.

FIG. 7. Genome editing activity in iPSCs and in the heart of an EGFP mouse model.

(a) VEsiCas activity in induced pluripotent stem cells (iPSCs). Percentage of non-fluorescent iPSC cells stably expressing EGFP after treatment with VEsiCas carrying either a control sgRNA (sgCtr) or a GFP targeting guide RNA (sgGFPI2). NT=not treated cells. Data presented as mean±s.e.m. for n=2 independent experiments. (b) VEsiCas mediated gene disruption in the cardiac muscle of EGFP-mice. Fluorescence microscopy images of cardiac tissue sections from EGFP transgenic mice 10 days after intra-cardiac injection of VEsiCas carrying a control sgRNA (sgCtr) or a guide targeting EGFP (sgEGFPBi). DAPI was used as a nuclear counterstain. Immunostaining for α-actinin was performed to identify cardiomyocytes (lower panels). A representative sample out of n=5 experiments is shown. Scale bar: 100 μm.

FIG. 8. Analysis of on-/off-target activity generated by VEsiCas on the VEGFA locus.

(a) Traceless delivery of SpCas9 through VEsiCas. Time course analysis of SpCas9 intracellular levels by western blot analysis in HEK293T cells transduced with VEsiCas or transfected with a plasmid expressing SpCas9. The reported time points correspond to the time of analysis following treatments (transduction or transfection). Western blot is representative of n=2 independent experiments. (b) Targeted comparison of genome editing specificity using VEsiCas or plasmid expressing SpCas9 in combination with sgVEGFA site3. Percentages of indels formation measured by TIDE in the VEGFA site3 locus and in two sgVEGFA site3 previously validated off-target sites (OT1 and OT3) in HEK293T cells. Dashed bar represents TIDE background. (c) Genome-wide specificity of VEsiCas targeting VEGFA site3 locus (nucleotides 4-26 of SEQ ID No.: 17). GUIDE-seq analysis for the sgRNA targeting VEGFA site3 locus in HEK293T cells transfected with SpCas9 or treated with VEsiCas. Black square indicates on-target sites. DNA from three biological replicates was mixed before library preparation. The right panel reports the total number of off-targets identified for the sgVEGFA site3 with SpCas9 transfection or VEsiCas treatments.

FIG. 9. Activation of gene expression by VEsiCas transcriptional regulator.

Activation of EGFP expression with VEsiCas-activator. Cells transfected with pTRE-EGFP reporter plasmids where treated with VEsiCas delivering promoter specific (sgTetO), or control (sgCtr) sgRNA, and dSpCas9-VP64. The graph shows percentage of EGFP positive cells two days after transduction.

FIG. 10. Delivery of gRNA molecules through VEsiCas.

VEsiCas derived from cells expressing sgRNAs targeting (sgEGFPBi) or not targeting (sgCtr) the EGFP locus were transduced into HEK293-EGFP cells, which were transfected with plasmid expressing SpCas9 24 hours before transduction. The graph shows percentage of EGFP negative cells seven days after transduction.

FIG. 11. Editing efficacy of VEsiCas loaded with myristylated SpCas9.

EGFP disruption assay in HEK293-EGFP cells using VEsiCas incorporating myristylated SpCas9 (myr-VEsiCas) and loaded with the sgEGFPBi guide RNA. The percentage of EGFP-negative cells obtained with the treatment is reported in the graph. Untreated cells serve as background control.

FIG. 12. Comparison of the editing efficacy of U6-vesicles and VEsiCas.

(a) EGFP disruption assay in HEK293-EGFP using either U6-vesicles or VEsiCas loaded with the sgEGFPBi guide RNA. The graph reports the percentages of EGFP-negative cells generated with each treatment, as indicated. Untreated cells serve as background control. (b) Relative editing efficacy of U6-vesicles with respect to VEsiCas obtained through the normalization of the data in (a) for the total amount of SpCas9 used to treat target cells.

FIG. 13. Comparison of the editing efficacy of Gesicles and VEsiCas.

Percentages of EGFP-negative cells generated after treatment of HEK293-EGFP with U6-vesicles, Gesicles or VEsiCas9, each loaded with the sgEGFPBi sgRNA. Cells were treated with a fraction of each preparation containing the same amount of SpCas9, as measured by western blot.

FIG. 14. Quantification of sgRNA incorporation into VEsiCas and U6-vesicles.

(a) Standard curve used for absolute sgRNA quantification by RT-qPCR obtained using an in vitro transcribed sgRNA identical to the one incorporated in each vesicle preparation. The equation of the trend line and the corresponding R-squared value are reported on the graph. (b) Total amount of sgRNA incorporated in U6-vesicles and VEsiCas measured by RT-qPCR using the standard curve in (a) for the interpolations. (c) Relative amount of sgRNA incorporated by U6-vesicles with respect to VEsiCas calculated from the normalization of the data in (b) for the total amount of SpCas9 present in each production, as quantified by western blot.

FIG. 15. Accurate quantification of SpCas9 incorporation into VEsiCas.

(a) Western blot of a representative VEsiCas preparation. Two different standard curves (Standard 1 and Standard 2), obtained with different preparations of recombinant SpCas9, are loaded together with the sample to allow quantification by densitometry. (b-c) Standard curves obtained from the densitometric analysis of the data in (a) used for absolute SpCas9 quantification. The equations of the trend line and the corresponding R-squared values are reported on each graph.

### DETAILED DESCRIPTION OF THE INVENTION

In the following description, numerous specific details are given to provide a thorough understanding of embodiments. The embodiments can be practiced without one or more of the specific details, or with other methods, components, materials, etc. in other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

RNA-guided nucleases, and in particular the CRISPR-Cas technology, are a powerful tool for genome editing and genome regulation. Its translation into clinical use strongly depends on further improvements in its specificity (complete abrogation of off-target activity) and delivery. These are tightly interdependent aspects of genome editing, since the amount and time of nuclease RNPs (e.g. gRNA and SpCas9) expression in recipient cells strongly correlate with the frequencies of off-target cleavages (Tsai & Joung 2016; Petris et al. 2017; Fu et al. 2013). Moreover, the persistence of SpCas9 expression may generate adverse immune responses towards the modified cells in vivo, further reinforcing the demand for a highly controllable method of delivery (Chew et al. 2016). Transient expression of SpCas9 has been obtained through physical and chemical (lipid- and polymer-based reagents) delivery methods (Zuris et al. 2015; Mout, Ray, Yesilbag Tonga, et al. 2017), which are not ideal for in vivo applications (Mout, Ray, Lee, et al. 2017). Premiere tools for in vivo gene delivery are viral vectors that however have serious limitations deriving from their potential risk of insertional mutagenesis (Chick et al. 2012; Petris et al. 2017) and permanent transgene expression that may increase the number of SpCas9 off-target cleavages (Petris et al. 2017). Genotoxicity associated with viral integration can be partially circumvented by using non-integrating viral vectors such as those deriving from adeno-associated viruses (AAV) (Zacchigna et al. 2014; Nakai et al. 2001; Ruozi et al. 2015). Due to size limitations, SpCas9 orthologues, such as the one from Staphylococcus aureus (Ran et al. 2015; Friedland et al. 2015), or split-proteins strategies (split-Cas9) have generally been employed with AAV vectors (Truong et al. 2015). Yet, the separation of CRISPR elements introduces high complexity into the system and occasional AAV integrations have been reported (Deyle & Russell 2009). Similarly, unpredictable insertional mutagenesis reported with other non-integrating vectors, such as the integrase-defective lentiviral vectors (IDLV), can be enhanced by nuclease activity beyond the background level (Gabriel et al. 2011; Wang et al. 2015). Attempts to combine the advantages offered by viral delivery together with transient SpCas9 expression were recently addressed by using a self-limiting CRISPR-Cas lentiviral vector (Petris et al. 2017), and by engineering lentiviral particles containing pre-packaged SpCas9 together with a non-integrating lentiviral vector expressing the gRNAs (Choi et al. 2016). A further step towards the exploitation of viral delivery properties in a DNA-free context is the development of viral-like particles (VLPs) (Mangeot et al. 2011; Voelkel et al. 2010). VLPs have been mainly used in the past decade for vaccination approaches (Ramqvist et al. 2007) and for the delivery of exogenous proteins (Mangeot et al. 2011; Voelkel et al. 2010).

In order to solve the above identified drawbacks of the known technology the present inventors were unexpectedly able to produce vesicles able to deliver—in a transient way—gRNAs and/or RNPs such as the one formed by a nuclease guided by a gRNA molecule (RNA-guided nuclease), whose gRNA component was abundantly packaged into vesicles exploiting a cytoplasmic expression system. The new surprising advantages include a much more efficient production and delivery of gRNAs or gRNA-nuclease RNPs, where the vesicle packaged nucleases are almost completely and correctly coupled with their gRNA due to the cytoplasmic transcription obtained in the appropriate permissive cells. Compared to nuclear expression of gRNAs the here described cytosolic expression system for gRNAs and RNPs packaging into cell released vesicles was several times more efficient. These vesicles are designed to have very minimal elements (e.g. viral elements such as VSV-g glycoprotein) necessary to trigger vesicles formation, the release of vesicles from producing cells, the efficient targeting and fusion of vesicles to target cells where delivery of transported gRNAs and/or RNPs have to occur.

The vesicles object of the instant application are designed to deliver gRNAs and/or gRNA-guided nuclease RNPs in a transient way. This is opposed to delivery through DNA transfection or viral vector delivery where the encoding DNA produces gRNAs and/or gRNA-guided nuclease continuously or at least till the encoding DNA is released by the cell (plasmid lost after cell division) which in turn will not occur following plasmid/viral vector integration into cellular chromatin. Conversely, RPN delivery results in an increased specificity, tolerability and safety of their applications in several fields (e.g. gene and genome editing and therapy, gene expression regulation, epigenetic modifications). The transient delivery minimizes risks due to: immune response against non-self elements present into vesicles; cleavage or modification of off-target site for gRNA and/or gRNA-nuclease RNPs and/or other of their associated elements present into vesicles; toxic effects due to the presence of vesicle components in particular gRNA and/or gRNA-nuclease RNPs, which can alter cellular homeostasis if expressed for a long time.

The present invention concerns a vesicle comprising:

i) a lipid envelope associated with at least one membrane-associated protein;

ii) at least one guide RNA molecule; and

iii) optionally at least one RNA-guided nuclease;

wherein the vesicle has at least one of the following features:
the at least one guide RNA molecule is present within the vesicle in an amount of at least 0.2% w/w to total vesicle protein mass; and
if the vesicle contains also the at least one RNA-guided nuclease, the at least one guide RNA molecule is complexed to the at least one RNA-guided nuclease, wherein the at least one guide RNA molecule is present within the vesicle in a molar ratio with respect to the at least one RNA-guided nuclease equal or above to 0.85:1.

In an embodiment, the lipid envelope is selected from a mono- or bi-layer lipid structure, an exosome, an enveloped virus, an enveloped viral-like particle, a microsome, an endosome, a nanosome, a vacuole. Preferably the lipid envelope is selected from an exosome, an enveloped virus or an enveloped viral-like particle; more preferably the lipid envelope is an enveloped viral-like particle.

In an embodiment, the at least one membrane-associated protein stimulates vesicle formation and/or mediates vesicle fusion to target cells. The at least one membrane-associated protein is selected from: Clatrin adaptor complex AP1, proteolipid protein PLP1, TSAP6, CHMP4C, VSV-G envelope protein, ALV envelope, BRL envelope glycoprotein, rabies virus envelope glycoprotein, influenza NA/HA/M2 envelope protein, MuLV amphotropic envelope, baculovirus gp64, HIV gp160, capsid proteins, nucleocapsid proteins, matrix protein of enveloped viruses having an interaction with the cell membrane, ebola VP40, ebola glycoprotein, Gag and/or Gag-pol retroviral protein, Gag and/or Gag-Pol lentiviral protein, Arc proteins, TY3/gypsy retrotransposons envelope proteins, HIV-1 Vpu, minimal-Gag, SADB19-VSV-G fusion, a portion of the VSV-G transmembrane domain and/or its intracellular domain and/or its extracellular domain fused with one of the envelope proteins listed above, single chain variable antibody fragments (scFv) derived from immunoglobulin variable domains able to recognize surface molecules on target cells, protein receptors able to recognize surface molecules on target cells, proteinaceous ligands able to recognize surface molecules on target cells and analogues thereof.

According to an embodiment, the at least one guide RNA molecule is selected from miRNA, shRNA, siRNA, sgRNA, crRNA, tracrRNA.

According to an embodiment, the at least one RNA-guided nuclease is selected from: CRISPR class 2 type-II, type-V, type-VI nucleases and Argonaute RNA-guided nucleases and variants thereof. Preferably, the at least one RNA-guided nuclease is selected from: a Cas9, Cpf1, Cas13 and Ago2 nuclease and variants thereof; a Cas9, Cpf1 and Cas13 nuclease mutant with or without nuclease activity; a Cas9 and Cpf1 nuclease mutant with nickase activity; a Cas9 and Cpf1 nuclease fused to a protein domain selected from: protein tags, additional nuclease domains, nucleic acid-editing domains, cell penetrating peptides and peptides allowing endosomal escape, transcriptional regulators, chromatin regulators, proteins or protein domains modulating DNA repair, proteins or protein domains allowing post-translational modification of other proteins, protein domains or peptides regulating protein stability and/or localization inside the cell, protein-binding domains. More preferably, the at least one RNA-guided nuclease is selected from: a Cas9, Cpf1 and Cas13 nuclease and variants thereof; still more preferably the at least one RNA-guided nuclease is selected from a Cas9 and Cpf1 nuclease and variants thereof.

According to one embodiment, the at least one guide RNA molecule, forming a complex with a Cas9, Cpf1, or Cas13 nuclease, or a Cas9, Cpf1 or Cas13 nuclease mutant with or without nuclease activity, is engineered to include an aptamer, preferably a MS2 aptamer, having interacting properties with an aptamer interacting protein domain, preferably a MS2 protein, which is fused to other protein domains encoding a base editor, a transcriptional regulator or a chromatin regulator. Preferably, the aptamer is included within the at least one guide RNA molecule at suitable positions (e.g. hairpin, nexus, tetraloop, stem) or at the 3′end of the at least one guide RNA molecule.

In an embodiment, the at least one RNA-guided nuclease is fused to at least one of: farnesylation signal, myristoylation signal, transmembrane domain.

The vesicle, once delivered to a target cell, provides for transient expression of the RNA-guided nuclease and/or guide RNA within the target cell in order to minimize cell toxicity.

Standard methods for gRNA expression are not suitable to produce the vesicles object of the instant invention in view of their poor efficiency of gRNAs and/or RNA-guided nucleases RNPs incorporation into vesicles. Efficient gRNA and/or RNA guided-nuclease delivery is of critical importance for genome editing using RNA-guided nucleases.

The present inventors therefore developed a new method to produce the vesicles object of the instant invention able to achieve high concentrations of gRNAs and/or RNA-guided nucleases RNPs in the cytoplasm for their packaging into vesicles.

The inventors have in fact surprisingly discovered that the efficient packaging of gRNAs and RNA guided-nuclease RNPs into cell-released vesicles requires high amount of gRNA localized in the cytoplasm of packaging cells and that dedicated biotechnological solutions are necessary to achieve this goal. This requirement is of extreme importance when gRNAs should not contain a 5′ cap, a poly-A sequence, must not be spliced by the cell machinery and/or processed by nuclear cellular enzymes (e.g. deamination, methylation, etc.). The requirement of a non-natural presence of high concentrations of gRNAs in the cytoplasm for efficient packaging into cell released vesicles can involve any kind of gRNA molecules, including gRNAs derived from processing of coding or non-coding RNAs, regardless of gRNA origin from a virus, an archaeal, bacterial, or eukaryotic cell (in particular if derived from bacteriophages or RNA viruses of eukaryotic cells replicating in the absence of, or out of, the cell nucleus). This aspect has critical importance for vesicle incorporation of gRNAs, which are usually transcribed by polymerases different from RNA polymerase-II (e.g. RNA polymerase-III) for biotechnological purposes, as disclosed for example in WO2015/191911. The inventors unexpectedly observed that standard nuclear Pol-III mediated expression of gRNAs does not allow their efficient release from the nucleus into the cell cytoplasm to allow their effective incorporation into cell-released vesicles.

The solution hereby disclosed by the inventors exploits direct cytoplasmic transcription and/or localization of gRNAs to overcome these limitations. gRNAs according to the invention are expressed by a natural or artificial system in the cell cytoplasm.

Surprisingly, this solution requires suitable permissive cells to express and localize high amounts of gRNA(s) and/or RNP(s) in the cytoplasm for their packaging into cell derived vesicles.

According to the present invention, the vesicles are produced by a method comprising the following steps:

i) providing a packaging cell, wherein the packaging cell has the following features:
a) the cell is tolerant to high levels of direct cytosolic transcription of at least one gRNA molecule and/or a complex formed by at least one gRNA molecule and at least one RNA-guided nuclease, wherein the cell tolerance is determined by: a lack of expression in the cell of at least one RNA virus-sensing pathway selected from: RIG-I, RIG-I-like protein, MDA-5, IPS-1, RIPI, FADD, TRAF6, TRAF3, TANK, NAP, NEMO, IKKα, IKKβ, IKKε, IKKγ, TBK1, DDX3, IκB, NF-κB, IRF3, IRF7, p65, p50, RIP1, TLR3, TLR7, TLR8, INF-α, INF-β, INF-γ1, INF-γ2, INF-γ3, Caspase-1; and/or a cell ability to cap at least one guide-RNA molecule by expression of capping enzymes; and/or a cell ability to express a 5′ phosphatase for de-phosphorylating 5′triphosphate transcripts; and
b) the cell stably or transiently expresses an RNA polymerase in the cytoplasm;

ii) transfecting the packaging cell with at least one first expression cassette, and at least one second expression cassette and optionally at least one third expression cassette, wherein:
a) the first expression cassette comprises a first nucleotide sequence to transcribe the at least one guide RNA molecule;
b) the second expression cassette comprises a second nucleotide sequence encoding the at least one membrane-associated protein; and
c) the third expression cassette comprises a third nucleotide sequence encoding the at least one RNA-guided nuclease;

iii) producing the vesicle from the packaging cells.

According to an embodiment, the RNA polymerase is a bacteriophage RNA polymerase selected from: RNA polymerase of phage T7, RNA polymerase of Bacteriophage SP6, RNA polymerase of Yersinia pestis bacteriophage phiA1122, RNA polymerase of Pseudomonas bacteriophage gh-1, RNA polymerase of Pseudomonas putida bacteriophage, RNA polymerase of Bacteriophage T3, RNA polymerase of Bacteriophage T4, RNA polymerase of Roseophage SIO1, RNA polymerase of Bacteriophage phiYe03-12, RNA polymerase of bacteriophage phiKMV, RNA polymerase of Enterobacteria bacteriophage K1-5, RNA polymerase of Vibriophage VpV262, RNA polymerase of BA14, RNA polymerase of BA127 and RNA polymerase of BA156, and variants thereof. More preferably, the RNA polymerase is selected from T7 RNA polymerase, SP6 RNA polymerase, T3 RNA polymerase, and T4 RNA polymerase. Still more preferably, the RNA polymerase is a T7 RNA polymerase.

In a further embodiment, the packaging cell is selected from BHK21, BSR-T7/5, BHK-T7 and Vero cells.

In an embodiment, the at least one membrane-associated protein, useful for stimulating vesicle formation and/or for mediating vesicle fusion to target cells, is selected from: Clatrin adaptor complex AP1, proteolipid protein PLP1, TSAP6, CHMP4C, VSV-G envelope protein, ALV envelope, BRL envelope glycoprotein, rabies virus envelope glycoprotein, influenza NA/HA/M2 envelope protein, MuLV amphotropic envelope, baculovirus gp64, HIV gp160, capsid proteins, nucleocapsid proteins, matrix protein of enveloped viruses having an interaction with a cell membrane, ebola VP40, ebola glycoprotein, Gag and/or Gag-pol retroviral protein, Gag and/or Gag-Pol lentiviral protein, Arc proteins, TY3/gypsy retrotransposons envelope proteins, HIV-1 Vpu, minimal-Gag, SADB19-VSV-G fusion, a portion of the VSV-G transmembrane domain and/or its intracellular domain and/or its extracellular domain fused with one of the envelope proteins listed above, single chain variable antibody fragments (scFv) derived from immunoglobulin variable domains able to recognize surface molecules on target cells, protein receptors able to recognize surface molecules on target cells, proteinaceous ligands able to recognize surface molecules on target cells and analogues thereof.

According to an embodiment, the at least one RNA-guided nuclease is selected from: CRISPR class 2 type-II, type-V, type-VI and Argonaute RNA-guided nucleases and variants thereof. Preferably, the at least one RNA-guided nuclease is selected from: a Cas9, Cpf1, Cas13 and Ago2 nuclease and variants thereof; a Cas9, Cpf1 and Cas13 nuclease mutant with or without nuclease activity; a Cas9 and Cpf1 nuclease mutant with nickase activity; a Cas9 and Cpf1 nuclease fused to a protein domain selected from: amino acid sequences that encode protein tags, additional nuclease domains, nucleic acid-editing domains, cell penetrating peptides and peptides allowing endosomal escape, transcriptional regulators, chromatin regulators, proteins or protein domains modulating DNA repair, proteins or protein domains allowing post-translational modification of other proteins, protein domains or peptides regulating protein stability and/or localization inside the cell, protein-binding domains. More preferably, the at least one RNA-guided nuclease is selected from a Cas9, Cpf1 and Cas13 nuclease and variants thereof; still more preferably the at least one RNA-guided nuclease is selected from a Cas9 and Cpf1 nuclease and variants thereof.

According to a further embodiment, the at least one guide RNA molecule, forming a complex with a Cas9, Cpf1 or Cas13 nuclease, or a Cas9, Cpf1 or Cas13 nuclease mutant with or without nuclease activity, is engineered to include an aptamer, preferably a MS2 aptamer, having interacting properties with an aptamer interacting protein domain, preferably a MS2 protein, which is fused to other protein domains encoding a base editor, a transcriptional regulator or a chromatin regulator. The aptamer can be included within sgRNA structure at suitable positions (e.g. hairpin, nexus, tetraloop, stem) or at the 3′end of the sgRNA.

According to a preferred embodiment, cell tolerance to direct cytosolic transcription of the gRNA is determined by: (i) a lack of expression in the cell of at least one RNA virus-sensing pathway selected from: RIG-I, RIG-I-like protein, MDA-5, IκB, NF-κB, IRF3, IRF7, INF-α, INF-13, INF-γ1, INF-γ2, INF-γ3; and/or (ii) a cell ability to cap at least one guide-RNA molecule by expression of capping enzymes; and/or (iii) a cell ability to express a 5′ phosphatase for de-phosphorylating 5′triphosphate transcripts. More preferably, the packaging cells are characterized by: (i) a lack of expression of at least one RNA-virus-sensing pathway selected from: RIG-I, INF-α, INF-β, INF-γ1, INF-γ2, INF-γ3; and/or (ii) a cell ability to cap at least one guide-RNA molecule by expression of capping enzymes; and/or (iii) a cell ability to express a 5′ phosphatase for de-phosphorylating 5′triphosphate transcripts.

According to the present invention, the method for producing the vesicles further provides in step ii) for transfecting the packaging cell with at least one further expression cassette, wherein the least one further expression cassette comprises a further nucleotide sequence encoding at least one membrane-associated protein, useful to change vesicles tropism to target cells, selected from: HIV gp160, HIV gp120, VSV-G, ALV envelope, ebola glycoprotein, BRL envelope glycoprotein, rabies virus envelope glycoprotein, influenza NA/HA/M2, MuLV amphotropic envelope, baculovirus gp64 TCR-alpha, CD4, MHC-I, MHC-II, variable domains of antibodies and/or full-length antibodies that recognize surface molecules on target cells, with the proviso that the further nucleotide sequence encodes at least one membrane-associated protein different from the at least one membrane-associated protein encoded by the second nucleotide sequence comprised in the second expression cassette. Preferably, the least one further expression cassette comprises a further nucleotide sequence encoding at least one membrane-associated protein selected from: BRL envelope glycoprotein, rabies virus envelope glycoprotein, SADB19-VSV-G fusion protein, a full length VSV-G protein or the cytosolic and transmembrane domains of the VSV-G protein fused to a receptor domain able to bind to a surface molecule on target cells. More preferably, the least one further expression cassette comprises a further nucleotide sequence encoding at least one membrane-associated protein selected from: BRL envelope glycoprotein, rabies virus envelope glycoprotein, SADB19-VSV-G fusion protein. In the following, definitions and further characteristics of the claimed features are provided.

Guide RNA Molecules

According to the present invention the at least one guide RNA molecule (gRNA) incorporated within the vesicles, which is used by RNA-guided nuclease(s) to bind the nucleic acid target, is selected from: sgRNA, crRNA, tracrRNA, miRNA, shRNA, siRNA.

The gRNA molecule can be either a natural or an artificial single guide RNA (sgRNA) or a guide RNA made by two or more transcripts.

Preferably, gRNAs can be bound by one or more proteins and/or other gRNAs molecules as described below.

The gRNA, used alone or in combination with other gRNAs, is able to bind and target the at least one RNA-guided nuclease, and in some embodiments also other associated molecules, to a nucleic acid target of interest (present in the target cell) at least in part complementary to the “guide” part of the gRNA molecule.

Some mismatches in the nucleotide sequences (e.g. 30%, 20%, 10%, 5%, 1%) between the guide portion of the gRNA molecule and the target nucleic acids might be present. The presence of mismatches can have an effect on the activity of the RNA-guided nuclease (e.g. the presence of mismatches can regulate nuclease activity, by triggering RNP binding of the target sequence but not its cleavage, or by influencing the cleavage rate and affinity such as in (Dahlman et al. 2015)).

Similarly, RNPs associated molecules can have their enzymatic function tightly regulated by the presence of mismatches between the gRNA and the target nucleic acid due to the different affinity and conformational changes involving the RNP components, target sequence and/or nearby regions.

The gRNA molecule can be modified to modulate nuclease affinity to the target of interest. The gRNA can be modified as described below (i) to be more or less stable compared to the parental natural or artificial gRNA, (ii) to abolish, reduce or increase its interaction with a target nucleic acid sequence and/or with one or more RNA-guided nuclease and/or associated molecules, (iii) to be traceable, (iv) to be further modified by in a mature or inactivated form by gRNA cleavage, (v) to be modified with post transcriptional modifications to add new functions to the original transcript or regulate its maturation.

The gRNA can be modified to specifically associate with other molecules and proteins to introduce new activity to the gRNA and/or the gRNA/RNA-guided nuclease complex. In particular, transcriptional activation or base editing can be obtained through the inclusion of an aptamer similar to the MS2 binding sequence into the gRNA which generates binding with an MS2 protein fused to transcriptional activation domains or base editing domains. Non-limiting examples of transcriptional activation domains are: VPR, VP64, VP16. Non-limiting examples of base editing domains are: nucleotide deaminase domains (e.g. cytidine deaminases (AID, APOBEC etc.) or adenine deaminases). The tethering of the transcriptional activation domains or base editing domains to or close to the RNA-guided nuclease target site generate transcriptional activation or base editing of the targeted gene.

The gRNA can be chemically modified by RNA deamination, incorporation of natural and artificial nucleotides.

The gRNA can be fused to: additional RNA domains either internally or at its 5′ and 3′ ends (e.g. 5′cap, MS2 repeats, RNA hairpins, fluorescent RNA aptamers (e.g. Broccoli, Spinach)), ribozymes, terminators, poly-A sequences.

The active gRNA can be constituted by the association of different RNA molecules, which interact by base pairing or other chemical link (e.g. covalent bond, hydrogen bond, salt bond), like for example the crRNA-tracrRNA dimer forming a functional gRNA molecule for some CRISPR-nucleases.

gRNA constant portions, defined also as scaffold, are portions of gRNAs not necessary for target selection, but required for binding by a RNA-guided nuclease or an associated molecule thereto (e.g. a protein, a second RNA).

gRNA scaffolds can be optimized to improve transcription, stability, folding and interaction with associated molecules, such as a RNA binding protein (e.g. (Thyme et al. 2016) and (Dang et al. 2015)). If the RNA binding protein is a SpCas9 molecule, the gRNA can be composed by a single gRNA and its scaffold (having a nucleotide sequence as set forth e.g. in SEQ ID No.: 44) can be optimized by mutations and nucleotide changes, as set forth e.g. in SEQ ID No.: 45 and 46 as described in (Dang et al. 2015).

For RNA-guided nucleases different from SpCas9 (e.g. other Cas9 orthologues, Cpf1 or Cas13 nuclease families) corresponding modifications of the cognate gRNA scaffold (e.g. by removal or mutation of T nucleotides to interrupt poly-T stretches and/or modification of the RNA hairpins structure and/or modification of the RNA loops) can be realized by a person skilled in the art in view of the common general knowledge.

T7 and SP6 RNA polymerase, which according to the invention can be used for cytoplasmic gRNA transcription, require an initial G, GG, GGG or GA sequence after their promoter to start transcription. Thus, in plasmids encoding the gRNA expression cassette such nucleotides can be added to the 5′ end of the transcript corresponding to the gRNA according to the invention by modifying the relative expression cassette to favor cytosolic transcription by such enzymes. Alternative polymerases could be used in substitution of T7 or SP6 RNA polymerase to obtain cytoplasmic transcription, for that reason the 5′-end sequence requirements to initiate transcription are changed accordingly.

Examples of gRNA modifications according to the invention are also described in U52015/0376586.

gRNA molecules useful within the present invention can be retro-transcribed to guide DNAs (gDNAs) before or after packaging into vesicles or before or after the fusion of the vesicle to target cells. Such gDNAs could be paired with DNA-guided nucleases for their targeting or used as donor DNAs.

gRNA(s) according to the invention can bind to other molecules, mutually influencing their activity and/or interactions. Such molecules include but are not limited to: proteins (e.g. MS2 protein), DNA (e.g. donor DNA molecules for HDR), and small molecules (e.g. small molecules for the regulation of a ribozyme, GTP, molecules binding to RNA aptamers (e.g. 3,5-difluoro-4-hydroxybenzylidene imidazolinone (DFHBI))).

RNA-Guided Nuclease(s)

The gRNA binds to a RNA-guided nuclease, which is a biologically relevant molecule guided to a target nucleic acid (e.g. RNA and/or DNA) by the gRNA. The RNA-guided nuclease can add a function to, or gain a function from, the interaction with the gRNA molecule.

In an embodiment, the RNA-guided nuclease is at least one protein which in combination with its gRNA could either simply bind to a target sequence or bring an activity to, contiguously to, or proximally to the target sequence according to its nuclease activity and/or to the presence of one or more additional molecules associated to the nuclease RNP(s) (examples of activities are described in details below).

According to the present invention such RNA-guided nucleases are selected from CRISPR associated nucleases and Argonaute RNA-guided nucleases, that is bacteriophage CRISPR-nucleases, Argonaute RNA-guided nucleases of bacterial origin or Argonaute RNA-guided nucleases of eukaryotic origin.

Preferably, the RNA-guided nuclease is selected from: CRISPR class 2 type-II, type-V, type-VI and Argonaute nucleases and variants thereof. More preferably, the RNA-guided nuclease is selected from: Cas9, Cpf1, Cas13 and Ago2 nucleases and variants thereof (as described below).

Activities of the nuclease RNP(s) can be on DNA, RNA or protein molecules found in spatial proximity (no more than 200 nm, 100 nm, 50 nm, 30 nm, 20 nm, 10 nm, 5 nm, 3 mn, 2 nm, 1 nm) to the target nucleic acid sequence, as determined by the gRNA, according to primary, secondary, tertiary and quaternary structure of the involved target molecules.

Examples of activities of the RNA-guided nuclease(s) to, contiguously to, or proximally to the target nucleic acid sequence include, but are not limited to: cleavage, nicking, binding, methylation, de-methylation, acetylation, de-acetylation, phosphorylation, de-phosphorylation, transcription activation, transcription repression, transcriptional interference, biotynylation, deamination, nucleotide deamination, cytosine deamination, guanosine deamination, translational interference, ubiquitinylation, ubiquitin-like modification, oxidation, reduction. Preferably these targets are therapeutically and/or diagnostically relevant DNA or RNA targets.

A Cas9 and/or a Cpf1 nuclease, as that term is used herein, refers to a nuclease that can interact with a gRNA molecule and, in concert with the gRNA molecule, localize (e.g., target or home) to a site which comprises a target domain and PAM sequence.

According to the present invention, a Cas9 nuclease from a variety of species can be used in the methods described herein. While within the experimental section of the present invention the S. pyogenes Cas9 nuclease has been used (having its amino acid sequences disclosed in UniProtKB ID Q99ZW2), Cas9 nucleases from the other species can exert the same activity.

Examples of species, from which Cas9 nucleases usable within the present invention can be obtained, include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheriae, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aures, Staphylococcus lugdunensis, Staphylococcus thermophilus, Streptococcus sp., Subdoligranulum sp., Tistrella mobilis, Treponema sp., or Verminephrobacter eiseniae. Preferably, the Cas9 nuclease is derived from Staphylococcus aureus (Chylinski et al. 2013), S. thermophilus and Neisseria meningitidis (Hou et al. 2013). More preferably, the Cas9 nuclease is derived from Staphylococcus pyrogenes. Naturally occurring Cas9 nucleases that can be used in the present invention include Cas9 nucleases of a cluster 1 bacterial family derived from: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clipl 1262), Enterococcus italicus (e.g., strain DSM 15952), or Enterococcus faecium (e.g., strain 1,231,408).

In some embodiments the Argonaute RNA-guided nuclease of eukaryotic origin is the RISC catalytic component Ago2, preferably mammalian or human Ago2.

RNA-guided nuclease variants, that can be used according to the present invention, are disclosed in the following.

In an embodiment, a Cas9 nuclease variant, e.g. a SpCas9 nuclease variant, comprises an amino acid sequence having at least 80%, 90%, preferably 95%, more preferably 98%, 99% or 100% identity with any Cas9 nuclease sequence recalled herein (and having its amino acid sequences as disclosed in UniProtKB ID Q99ZW2) or a naturally occurring Cas9 nuclease sequence derived from the above listed species.

In some embodiments the Cas9 nuclease can also be an engineered Cas9 nuclease (i.e. a Cas9 nuclease variant) as disclosed i.a. in Nunez et al. 2016; Wright et al. 2015; Zetsche et al. 2015; Nihongaki et al. 2015; Kleinstiver et al. 2016; Slaymaker et al. 2016; Italian patent application no. 102017000016321; WO2016/205613 and WO2017/040348.

The Cas9 nuclease can also be mutated to be a nickase (e.g. SpCas9 D10A or H840A mutants with respect to the reference sequence as disclosed in UniProtKB ID Q99ZW2), or to be unable to cleave the DNA (e.g. mutating the amino acid positions D10A/D839A/H840A, or D10A/D839A/H840A/N863A with respect to the reference sequence as disclosed in UniProtKB ID Q99ZW2). In some embodiments the RNA-guided nuclease is fused to amino acid sequences, which can provide new functions, localizations, detectabilities, regulatory activities and other effects known to a person skilled in the art. The amino acid sequences that can be fused to the RNA-guided nuclease is selected from: protein tags, additional nuclease domains, nucleic acid-editing domains, cell penetrating peptides and peptides allowing endosomal escape, transcriptional regulators, chromatin regulators, proteins or protein domains modulating DNA repair, proteins or protein domains allowing post-translational modification of other proteins, protein domains or peptides regulating protein stability and/or localization inside the cell, protein-binding domains. Preferably the amino acid sequences that can be fused to the RNA-guided nuclease is selected from: protein tags, additional nuclease domains, nucleic acid-editing domains, transcriptional regulators, chromatin regulators, protein domains or peptides regulating protein stability and/or localization inside the cell.

These modifications are detailed in the following paragraphs.

In some embodiments the RNA-guided nuclease is fused to protein tags (e.g. V5-tag, FLAG-tag, myc-tag, HA-tag, GST-tag, polyHis-tag, MBP-tag, SUN-tag, full length or part of EGFP protein or similar fluorescent proteins) useful for facilitate the detectability of the RNA-guided nuclease.

In some embodiments the RNA-guided nuclease is fused to additional nuclease domain(s) (e.g. Fok-I or another RNA-guided nuclease) to increase specificity or activity.

In some embodiments the RNA-guided nuclease is fused to a nucleic acid-editing domain acting on DNA or RNA such as for example adenosine deaminase or cytidine deaminase (e.g. AID, APOBEC).

In some embodiments the RNA-guided nuclease can be fused to cell penetrating peptides (e.g. poly arginine peptides) or other molecules favoring endosomal escape (e.g. ppTG21 as described in (Rittner et al. 2002)).

In some embodiments the RNA-guided nuclease can be fused to at least one protein which could influence gene expression or alter DNA repair such as: transcriptional activators (e.g. VP16, VP64, VPR), transcriptional repressors (e.g. KRAB), inhibitors of DNA repair (GAM, UGI)); guanosyl transferase, DNA methyltransferase (e.g. DNMT1; DNMT3), RNA methyltransferases, DNA demethylases, RNA demethylases acetyltransferases, deacetylase, ubiquitin-ligases, deubiquitinases, kinases, phosphatases, NEDD8-ligases, de-NEDDylases, SUMO-ligases, deSUMOylases, histone deacetylases (e.g. HDAC), histone acetyltransferases (e.g. p300), histone methyltransferases, histone demethylases), protein DNA binding domains (e.g. zinc finger domain or TALE), RNA binding proteins (e.g. MS2 protein).

In some embodiments the RNA-guided nuclease is fused to additional protein domains and/or peptide sequences regulating localization or stability of the RNA-guided nuclease such as: full length or portions of Gag protein, retroviral or lentiviral Vpr protein, Cyclophilin A protein, nuclear localization signals (e.g. SV40 nuclear localization signal, c-Myc NLS, PY-NLSs, bipartite nuclear localization signals as described in (Suzuki et al. 2016)), nuclear export signals (e.g. HIV-1 Rev nuclear export signal), mitochondrial localization signals, plastid localization signals, subcellular localization signals, membrane targeting signals (e.g. leader peptides), lipidation signals (e.g. myristoylation signals (consensus sequence: M-G-X-X-X-S), palmytoylation signal, prenylation signal, isoprenylation signal, destabilizing degrons signals (e.g. Geminin destruction box motifs).

In some embodiments the RNA-guided nuclease can be fused to protein binding signals such as: dimerization or multimerization domains, intrabodies (e.g. anti-SUN-tag intrabody), a half of a split protein or biological tethering domains (e.g. MS2, Csy4 and lambda N protein).

According to the invention a RNA-guided nuclease can be reconstituted from one or more fragment thereof; preferably whereby an intein or a protein intron or a dimerizing domain is included within the RNA-guided nuclease (e.g. (Truong et al. 2015)). Preferably, such fragments can be induced to reconstitute a catalytically active RNA-guided nuclease protein by intein dimerization of a split-Cas9 as disclosed e.g. in (Truong et al. 2015).

In some embodiment the reconstituting step can be performed in vitro, in some other embodiment it can be performed in vivo (see references below). Preferably, such fragments can be induced to reconstitute the RNA-guided nuclease similarly to Cas9 nuclease by dimerization of a split-Cas9 as disclosed e.g. in (Wright et al. 2015) and (Liu et al. 2016).

According to the invention a Cas9 nuclease can be engineered to recognize a PAM sequence different from the one targeted by the wild type Cas9 nuclease. In some embodiments the Cas9 nuclease can be engineered to recognize relaxed or new PAM sequences. Cas9 variants may comprise one or more additional mutations at residues D1135V/R1335Q/T1337R (QVR variant), D1135E/R1335Q/T1337R (EVR variant), D1135V/G1218R/R1335Q/T1337R (VRQR variant), D1135V/G1218R/R1335E/T1337R (VRER variant) of the amino acid sequence disclosed in UniprotKB ID Q99ZW2, as disclosed in the US patent application US2016/0319260. In some embodiments a Cas9 nuclease can be modified to recognize a new or a relaxed PAM sequence (A262T/R324L/S409I/E480K/E543D/M694I/E1219V mutations relative to the amino acid sequence disclosed in UniprotKB ID Q99ZW2), while having also mutation improving its specificity (e.g. xCas9-3.6 or xCas9-3.7 as disclosed in (Hu et al. 2018)).

According to the invention any other Cas9 or RNA-guided nuclease variant known in the art targeting different PAMs coupled with a gRNA transcribed in the cytoplasm of permissive cells can be used.

A Cas9 nuclease can be further modified according to the invention to be targeted to membranes or to endosome as disclosed i.a. in WO2015/191911.

A Cas9 nuclease can be substituted by a different subtype and class of RNA-guided nucleases targeting DNA including but not limited to type V CRISPR-associated RNA-guided nucleases. Preferably, these type V CRISPR-associated nuclease are Cpf1 nucleases (e.g. Acidaminococcus sp. Cpf1 (AsCpf1), Lachnospiraceae Bacterium Cpf1 (LbCpf1), Alicyclobacillus acidoterrestris C2c1 (AacC2c1)) as disclosed in (Shmakov et al. 2015).

The Cpf1 RNA-guided nucleases can be modified to function beyond DNA cleavage (as disclosed in (Tak et al. 2017)). Some non-limiting examples of Cpf1 nuclease variants are: AsCpf1 containing the mutation R1226A (referred to the amino acid sequence disclosed in UniprotKB ID U2UMQ6) to obtain a DNA nickase; the LbCpf1 D832A/E925A (referred to the amino acid sequence disclosed in PDB ID 5ID6_A) mutant unable to cleave DNA; the fusion with other polypeptides having or not having a catalytic activity similarly to Cas9 fusions (e.g. VPR, KRAS transcriptional regulators, p300 acetylase core, APOBEC, AID etc) as disclosed for example in (Tak et al. 2017).

In some embodiments, Cpf1 nucleases can be modified to target different PAMs (as disclosed in (L. Gao et al. 2017)) and function beyond DNA cleavage (e.g. used in combination with other protein domains such as adenine or cytidine deaminase) similarly to Cas9 nuclease-dead engineered variants.

RNA-guided nucleases useful within the present invention are also represented by RNA-guided nucleases targeting RNA including, but not limited to, type VI CRISPR-associated RNA-guided RNase Cas13 (e.g. Cas13a (previously known as C2c2), Cas13b, Cas13c and Cas13d (Yan et al. 2018)) and Argonaute nucleases guided by RNA (e.g. CRISPR-associated Marinitoga piezophila Argonaute-gRNA (Lapinaite et al. 2018), RISC catalytic component Ago2).

As mentioned above for Cas9, the Cas13 or Ago2 RNA-guided nucleases targeting RNA can be similarly modified to function beyond RNA cleavage (as disclosed in (Cox et al. 2017)).

Vehicles to Deliver gRNA(s) and RNP(s) According to the Invention

Vehicles suitable for the delivery of said gRNAs and RNPs are vesicles which, according to the invention, are cytoplasm-derived structures released by a cell spontaneously and/or after internal and/or external stimulation.

Vesicles released form a cell can be, but are not limited to: any membrane formed vesicle enclosed by a lipid envelope naturally and/or artificially released from a cell having diameter between 1000 and 5 nm. Examples of membrane formed vesicles released from a cell are exosomes, enveloped viruses (e.g. Herpesviridae, Coronaviridae, Hepadnaviridae, Poxviridae, Retroviridae, Paramyxoviridae, Arenaviridae, Filoviridae, Bunyaviridae, Orthomyxoviridae, Togaviridae, Flaviviridae, Hepatitis D virus), viral-like particles, endogenous or ancestral viral-like particles, microsomes, endosomes, nanosomes, vaquoles, multivesicular bodies.

Preferably, vesicles are exosome-like particles or viral-like particles, with a size of 10-1000 nm, 20-500 nm, 40-400 nm, or 70-300, more preferably a size of 80-200 nm.

Vesicles according to the invention are engineered to include said gRNA and gRNA-associated nucleases.

Vesicles according to the invention contain at least one guide RNA molecule present within the vesicles in an amount of at least 0.2% w/w to total vesicle protein mass.

If the vesicle according to the invention contains also the at least one RNA-guided nuclease, the at least one guide RNA molecule is complexed to the at least one RNA-guided nuclease, wherein the at least one guide RNA molecule is present within the vesicle in a molar ratio with respect to the at least one RNA-guided nuclease in a range 0.85:1 to 10:1.

Cytoplasmic Transcription Systems to Accumulate RNAs into the Cytosol

Preferably transcription systems for the direct expression of RNA into the cytoplasm are exogenous to the cytoplasm of producing cell.

These systems include RNA polymerases from mitochondria, other eukaryotic organism, but also viral and bacterial RNA polymerases, which can be either derived from pathogens of the producing cell or from completely unrelated microorganisms.

The RNA polymerase of choice according to the present invention is selected from: RNA polymerase of phage T7, RNA polymerase of Bacteriophage SP6, RNA polymerase of Yersinia pestis bacteriophage phiA1122, RNA polymerase of Pseudomonas bacteriophage gh-1, RNA polymerase of Pseudomonas putida bacteriophage; RNA polymerase of Bacteriophage T3, RNA polymerase of Bacteriophage T4, RNA polymerase of Roseophage SI01, RNA polymerase of Bacteriophage phiYe03-12, RNA polymerase of bacteriophage phiKMV, RNA polymerase of Enterobacteria bacteriophage K1-5, RNA polymerase of Vibriophage VpV262, RNA polymerase of BA14, RNA polymerase of BA127 and RNA polymerase of BA156, and variants thereof.

Variants of RNA polymerases that can be used within the present invention can be identified using the Conserved Domain Architecture Retrieval Tool (CDART) program of the National Center for Biotechnology Information (Geer et al. 2002) or by similar or other predictive programs, which use T7 or SP6 RNA polymerase as model sequence. Examples of enzymes identified in this manner include: T odd bacteriophages or related viruses including Enterobacteria. Preferably, the RNA polymerase is selected from RNA polymerase of phage T7, RNA polymerase of Bacteriophage SP6, RNA polymerase of Bacteriophage T3, RNA polymerase of Bacteriophage T4, and variants thereof. More preferably, the RNA polymerase is selected from RNA polymerase of phage T7, RNA polymerase of Bacteriophage SP6, and variants thereof.

In several applications, T7 RNA polymerase can be substituted by another phage-derived RNA polymerase (e.g. SP6 RNA polymerase) with some obvious modifications in the expression system (e.g. change of promoter and/or terminator sequences).

The T7 or SP6 RNA polymerase variants have nucleotide sequences having at least 60%, 70%, 80%, 90%, 95%, 98%, 99% or 100%, preferably 90%, 95%, 98%, 99% or 100%, identity to wild-type T7 or SP6 RNA polymerase with SEQ ID N: 40 and 42, respectively. The T7 or SP6 RNA polymerase has an amino acid sequence with at least 80%, 90%, 95%, 98%, 99% or 100% sequence identity to SEQ ID NO: 41 and 43, respectively.

Mutants of phage polymerase can be used according to the invention. Such mutants have the ability to transcribe from modified promoters, and to transcribe starting with different nucleotides (e.g. T7 RNA polymerase starting transcription from a GA or AA, instead of canonical GG or GGG as disclosed in (Esvelt et al. 2011)).

Preferably the artificial cytoplasmic transcription of the invention for the incorporation of transcript gRNAs and gRNA-nuclease RNPs into vesicles is performed in a eukaryotic cell (human, mammalian, insect, plant or yeast cell) from which such vesicles can be derived. However, cytoplasmic transcription according to the invention can be achieved also in prokaryotic and archea cells. More preferably the eukaryotic cell is a mammalian cell. Most preferably is a mouse, hamster, human or a non-human primate cell.

Of note, producing cells of vesicles according to the invention has to tolerate expression of transcript produced by the cytosolic transcriptional system (e.g. T7 or SP6 RNA polymerase). Such ectopic production of transcripts can be toxic and trigger an antiviral cellular response (e.g. interferon response) in the producing cells, which is detrimental for the ectopic transcription and also for endogenous transcription and translation in the producing cells. Indeed, T7 or SP6 RNA polymerase produces uncapped 5′ triphosphate transcripts with non-self features (e.g. 5′ diphosphate RNA, ssRNA, dsRNA). Such transcripts are detected in most cell types by innate cellular immunity, which induces as consequence a non-permissive antiviral state, dampening further cytoplasmic transcription, blocking the translation of several proteins and strongly altering the physiological state of the cell. Only cells deficient in one or more of their antiviral pathways can be used in combination with the T7 RNA polymerase or related enzymes to accumulate high amounts of the transcripts of interest in the cytoplasm of mammalian or eukaryotic cells.

In view of the above, the packaging cells useful according to the present invention are characterized by: (i) a lack of expression of at least one RNA-virus-sensing pathway selected from: RIG-I, RIG-I-like protein, MDA-5, IPS-1, RIPI, FADD, TRAF6, TRAF3, TANK, NAP, NEMO, IKKα, IKKβ, IKKε, IKKγ TBK1, DDX3, IκB, NF-κB, IRF3, IRF7, p65, p50, RIP1, TLR3, TLR7, TLR8, INF-α, INF-β, INF-γ1, INF-γ2, INF-γ3, Caspase-1; and/or (ii) a cell ability to cap at least one guide-RNA molecule by expression of capping enzymes; and/or (iii) a cell ability to express a 5′ phosphatase for de-phosphorylating 5′triphosphate transcripts. Preferably, the packaging cells are characterized by: (i) a lack of expression of at least one RNA-virus-sensing pathway selected from: RIG-I, RIG-I-like protein, MDA-5, IκB, NF-κB, IRF3, IRF7, INF-α, INF-β, INF-γ1, INF-γ2, INF-γ3; and/or (ii) a cell ability to cap at least one guide-RNA molecule by expression of capping enzymes; and/or (iii) a cell ability to express a 5′ phosphatase for de-phosphorylating 5′triphosphate transcripts. More preferably, the packaging cells are characterized by: (i) a lack of expression of at least one RNA-virus-sensing pathway selected from: RIG-I, INF-α, INF-β, INF-γ1, INF-γ2, INF-γ3; and/or (ii) a cell ability to cap at least one guide-RNA molecule by expression of capping enzymes; and/or (iii) a cell ability to express a 5′ phosphatase for de-phosphorylating 5′triphosphate transcripts.

This is an essential requirement for cells suitable to be used as efficient producing cells for vesicles containing RNAs and RNPs produced by cytoplasmic transcription. It is known to the art the existence of cells which are defective in the pathways involving these proteins and are thus not, or less, affected by the accumulation non-self cytosolic transcription products.

Example of cells deficient in at least one of the RNA virus-sensing pathway as listed above are BHK21, BSR-T7/5, BHK-T7 (Eaton et al. 2017) and VERO cells.

These cells can be modified to stably or transiently express the cytoplasmic transcriptional system (Eaton et al. 2017).

A reproducible method to obtain producing cells of vesicles according to the invention is to transfect BHK21 or VERO cells with plasmids, or transduce with viral vectors encoding T7-RNA polymerase and optionally a selectable marker (e.g. puromycin resistance or EGFP), according to obvious procedures of molecular biology know to an average person skilled in the art.

Other common mammalian, chicken and insect cells (e.g. HEK-293, HEK-293T, Hela, U20S, iPSC, DT40, HighS, Sf9) can be treated or modified to be permissive for efficient cytoplasmic transcription by (i) genetic knock-out (e.g. using targeted nucleases), (ii) transcriptional interference (e.g. siRNA and shRNA), or (iii) pharmacological inhibition (e.g. interferon inhibitors, NF-κB inhibitors, IκB/IKK inhibitors) of the above mentioned pathways and proteins, known to be involved in response to non-self and viral transcripts. Insect cells present several advantages. In particular, they are devoid of undesirable human proteins, and their culture does not require animal serum.

It is also possible to circumvent the requirement of a permissive cell deficient in the RNA virus-sensing pathways mentioned above, by engineering the producing cell to express one or more capping enzymes in the cytosol. Such capping enzymes could be directly or indirectly fused to the RNA polymerase used for cytosolic transcription. One non limiting example is the fusion of T7 RNA polymerase with African Swine Fever Virus NP868R Capping Enzyme (Eaton et al. 2017). Another non-limiting example is the capping enzyme activity of vaccinia virus enzymes (Dl and D12 to form cap-0 structure and VP39 to form cap-1 structure), which allows cytosolic RNA transcription by T7 RNA polymerase in cells poorly permissive in the absence of vaccinia infection to express capping proteins (Elroy-Stein & Moss 2001; Fuchs et al. 2016). In non-permissive cells (e.g. HEK-293), however, obtaining very high levels of efficiently 5′capped cytosolic T7 RNA polymerase expressed transcripts requires the infection with live vaccinia virus which has enzymes able to form cap-1 structures on RNA expressed directly in the cytoplasm (Wei & Moss 1975).

A cell tolerant to cytosolic transcription could be also obtained through expression of a 5′-phosphatase which performs de-phosphorylation of 5′-triphosphate cytosolic transcripts.

Methods to Stimulate Vesicles Formation

Vesicles are spontaneously released by cells, however this natural process produces vesicle titres usually too low for reliable industrial exploitation.

Production of vesicles according to the invention can be artificially enhanced to obtain much more efficiently particles incorporating gRNAs and RNA-associated nucleases according to the invention.

Stimulation of the cell to release vesicles can be realized by employing vesicle inducers selected from: incorporation within the vesicle of at least one membrane-associated protein, physical methods and/or chemical compounds.

According to one embodiment, an increase in the formation of existing cell-released vesicles or the induction of the production of new cell-released vesicles can be achieved through expression of at least one membrane-associated protein, wherein the at least one membrane-associated protein is encoded by the second nucleotide sequence contained in the second expression cassette used in the transfection step ii) of the vesicle producing method disclosed herein.

A non-limiting list of the at least one membrane-associated protein encoded by the second expression cassette includes: cellular proteins involved in vesicle formation (e.g. Clatrin adaptor complex AP1, proteolipid protein PLP1, TSAP6, CHMP4C); viral structural proteins of enveloped viruses (e.g. VSV-G envelope protein, ALV envelope, BRL envelope glycoprotein, rabies virus envelope glycoprotein, influenza NA/HA/M2, MuLV amphotropic envelope, baculovirus gp64, HIV gp160; capsid proteins, nucleocapsid proteins, matrix protein of enveloped viruses having an interaction with the cell membrane; ebola VP40, ebola glycoprotein, Gag and/or Gag-pol retroviral protein, Gag and/or Gag-Pol lentiviral protein); endogenous or ancestral retroviral-like proteins (Arc proteins, TY3/gypsy retrotransposons env proteins); viral non-structural proteins (HIV-1 Vpu); artificial proteins (minimal-Gag (Accola et al. 2000), SADB19-VSV-G fusion (Schoderboeck et al. 2015)); part of the VSV-G transmembrane domain and/or its intracellular domain and/or its extracellular domain fused with one of the envelope proteins listed above or single chain variable antibody fragments (scFv) derived from immunoglobulin variable domains or protein receptors or proteinaceous ligands able to recognize surface molecules on target cells. Preferably, the at least one membrane-associated protein is selected from: clatrin adaptor complex AP1, proteolipid protein PLP1, TSAP6, CHMP4C, VSV-G envelope protein, ebola VP40 envelope protein, ebola glycoprotein, BRL envelope glycoprotein, rabies virus envelope glycoprotein, influenza NA/HA/M2 envelope protein, MuLV amphotropic envelope protein, baculovirus gp64 envelope protein, part of the VSV-G transmembrane domain and/or its intracellular domain and/or its extracellular domain fused with one of the envelope proteins listed above. More preferably, the at least one membrane-associated protein is selected from: VSV-G envelope protein, ebola VP40 envelope protein, ebola glycoprotein, BRL envelope glycoprotein, rabies virus envelope glycoprotein, influenza NA/HA/M2 envelope protein, MuLV amphotropic envelope protein, baculovirus gp64 envelope protein; still more preferably the at least one membrane-associated protein is the VSV-G envelope protein.

Additionally, several kinds of cell-damaging chemical or physical treatments can be used to trigger the release of extracellular vesicles from cells which can be used to package gRNAs and RNA-guided nucleases according to the invention. A non-limiting list of such physical and/or chemical methods include photodynamic stimulation (i.e. Foscan® m-THPC (5,10,15,20-tetra(3-hydroxyphenyl)chlorin) and light stimulation similar to what described in (Aubertin et al. 2016)), ionizing radiation, heat stress (Bewicke-Copley et al. 2017), change in culture conditions, calcium and/or salts concentrations, doxorubicin (Aubertin et al. 2016), cisplatin (Samuel et al. 2018).

Methods to Enrich the Loading of gRNA and/or gRNA/RNA-Guided Nuclease Complexes in Vesicles According to the Invention.

A gRNA according to the invention can be more efficiently packaged into cell released vesicles if it can be locally enriched in the cytosol in proximity to the plasma membrane or in proximity to the Golgi, ER, nuclear, mitochondria, peroxisome, endosome membranes, where vesicles released by cells are often formed.

A RNA-guided nuclease can be engineered to capture gRNA molecules according to the invention and to enrich their relative abundance in a cytosolic subcellular compartment or area. In the art, methods are described to enrich protein localization near cellular membranes (see e.g WO2015/191911). Such methods include the use of membrane targeting or membrane affinity signals (i.e. direct fusion with one or more farnesylation and/or myristoylation signals, one or more transmembrane domains or endosome). For example the Cas9 protein can be modified to be targeted to transmembrane domains or to endosome as disclosed i.a. in WO2015/191911.

If the protein binds to gRNAs, it can be used to increase gRNA loading into vesicles. Interaction between the gRNA and membrane targeting proteins can be direct or mediated by a protein carrier or factor (i.e. methods described in patents WO2014200659A1 and PCT/EP2010/067200).

Vesicle Purification and Enrichment

Vesicles according to the invention can be administered after purification or through co-culture of producing cells with target cells (either in contact or separated by a selective size excluding membrane to allow passage of the vesicles). In addition, vesicles-producing cells may be engrafted into an human, non-human primate, mouse, rat, fish organism to release vesicles directly in vivo.

Purification steps are described in a non-limiting way in the results section. Several published protocols for the enrichment of cell-released vesicles and viruses can be applied for vesicle purification: i.e. filtration through 0.45 or 0.22 um pore filter, use of affinity tags or proteins present on vesicles surface (biotin-streptavidin, Ag-mAb), centrifugation in the absence or presence of a sucrose cushion (i.e. 20% sucrose, 10% sucrose, 5% sucrose), a sucrose gradient (5-60%, 10-60%, 20-60%), use of polyethylene glycole polymers (PEG-4000, PEG6000).

Cell Targeting and Fusion Properties

Cell targeting and/or fusion of vesicles according to the invention can be obtained by incorporating within the vesicles at least one membrane-associated protein as discussed above in section “Methods to stimulate vesicle formation”.

To increase the cell targeting and/or fusion properties of the vesicles (i.e. vesicle tropism and cellular fusion properties), the vesicles can contain at least one further membrane-associated protein. Such at least one further membrane-associated protein is expressed through a further expression cassette used in the transfection step ii) of the vesicle producing method disclosed herein. The further expression cassette contains a further nucleotide sequence encoding the at least one further membrane-associated protein, provided that such at least one further membrane-associated protein is different from the at least one membrane-associated protein encoded by the second nucleotide sequence contained in the second expression cassette.

The at least one further membrane-associated protein is selected from: HIV gp160, HIV gp120, VSV-G, ALV envelope, ebola glycoprotein, BRL envelope glycoprotein, rabies virus envelope glycoprotein, influenza NA/HA/M2, MuLV amphotropic envelope, baculovirus gp64. Additional membrane-associated proteins suitable to direct vesicle to intended target cells are membrane receptors and/or ligands (e.g. TCR-alpha, CD4, MHC-I, MHC-II), variable domains of antibodies and/or full-length antibodies that recognize surface molecules on target cells directly or indirectly linked to the cell membrane (i.e. monospecific and bispecific antibodies, SIP, minibodies, nanobodies, heavy-chain antibodies, single-domain antibodies). Preferably, the at least one further membrane-associated protein is selected from: HIV gp160, HIV gp120, VSV-G, ALV envelope, ebola glycoprotein, BRL envelope glycoprotein, rabies virus envelope glycoprotein, influenza NA/HA/M2, MuLV amphotropic envelope, baculovirus gp64, TCR-alpha, CD4, MHC-I, MHC-II.

The budding, targeting and fusion activity of the vesicles according to the invention can result from the activity of a single (i.e. VSV-G) or multiple different proteins (i.e. mimicking natural or artificial pseudotyped viruses and vectors).

Vesicles according to the invention can target virtually any cell where the delivered gRNA and/or RNA-guided nuclease RNPs can have a biological effect. Preferably such effect involves the binding and/or cleavage of a nucleic acid present in the target cell.

### Preferred Embodiment

In the following, a discussion about a preferred embodiment of the instant description is provided, wherein the vesicles (named VEsiCas) are able to deliver CRISPR-Cas components employing as membrane-associated protein constituting the vesicle envelope the VSV-G protein. Such data are not limiting with respect to the actual scope of the present invention as claimed in the pending set of claims.

The present inventors discovered that vesicles incorporating VSV-G protein, in combination with SpCas9, were sufficiently loaded with SpCas9 protein. However, vesicles produced under standard experimental conditions using nuclear U6-based transcription for the sgRNA synthesis showed poor genome editing activity, suggesting non-sufficient amounts of incorporated sgRNA. This limitation was circumvented by favoring SpCas9 protein assembly with the sgRNA in producing cells through cytoplasmic sgRNA synthesis driven by the T7 RNA polymerase. To this end the present inventors employed cells resistant to the cytotoxicity induced by high levels of uncapped 5′-triphosphate cytoplasmic RNA, BSR-T7/5 (Habjan et al. 2008). This cell line is deficient for the RNA sensing RIG-I pathway, leading to interferon activation, and is stably transfected to express the T7 RNA polymerase.

A remarkable advantage offered by the VEsiCas approach is the transient nature of delivered SpCas9. As demonstrated in this study the rapid clearance of SpCas9, which decreased as soon as 12 hours post VEsiCas treatment, strongly lowered the off-target activity associated with genome editing, as opposed to high levels of non-specific cleavages generated by plasmid transfected SpCas9. The present data also prove that VEsiCas are more efficient in delivering Cas9 RNP complexes than electroporation. In fact, the present system requires less SpCas9 protein, thus offering a clear advantage in preventing potential adverse effect of immune responses against the edited cells (Chew et al. 2016). Finally, VEsiCas can be readily adapted to more complex genome editing approaches, such as the use of Cas9 nickase, requiring incorporation of sgRNAs pairs (Komor et al. 2017).

Overall, the efficient and traceless delivery of CRISPR-Cas9 through the VEsiCas approach represents a further advancement towards safer in vivo genome editing.

Results

Design and Development of VSV-G Enveloped SpCas9-sgRNA Vesicles, VEsiCas

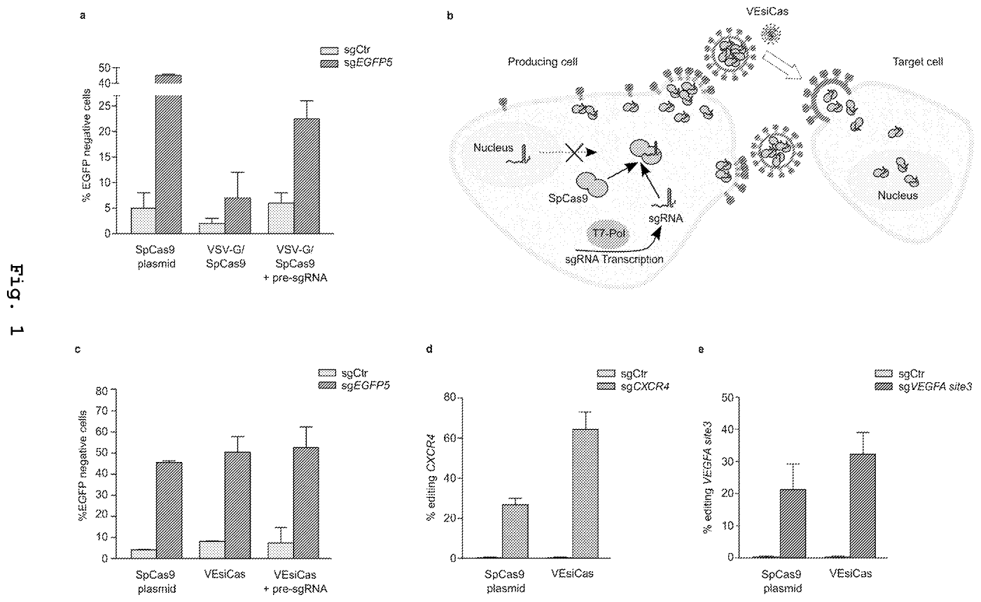

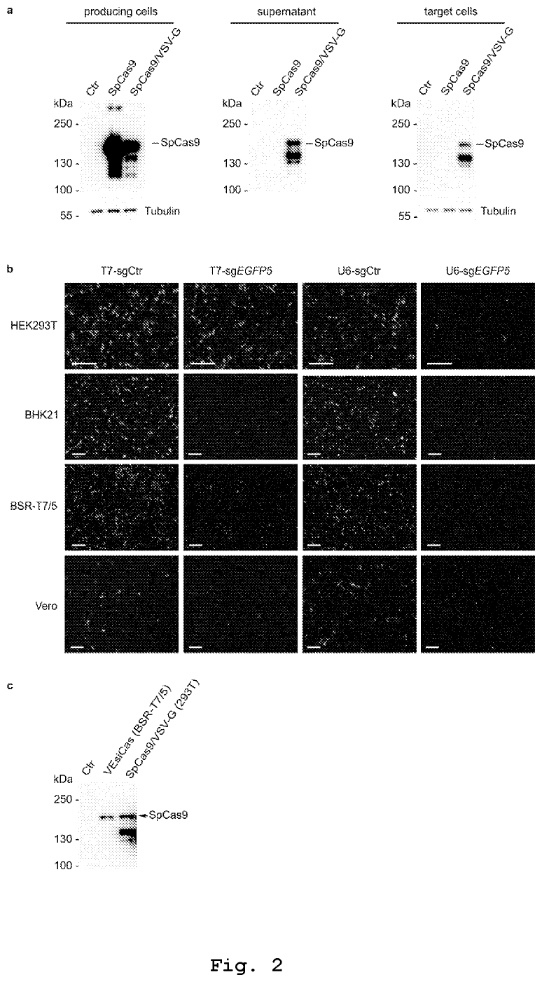

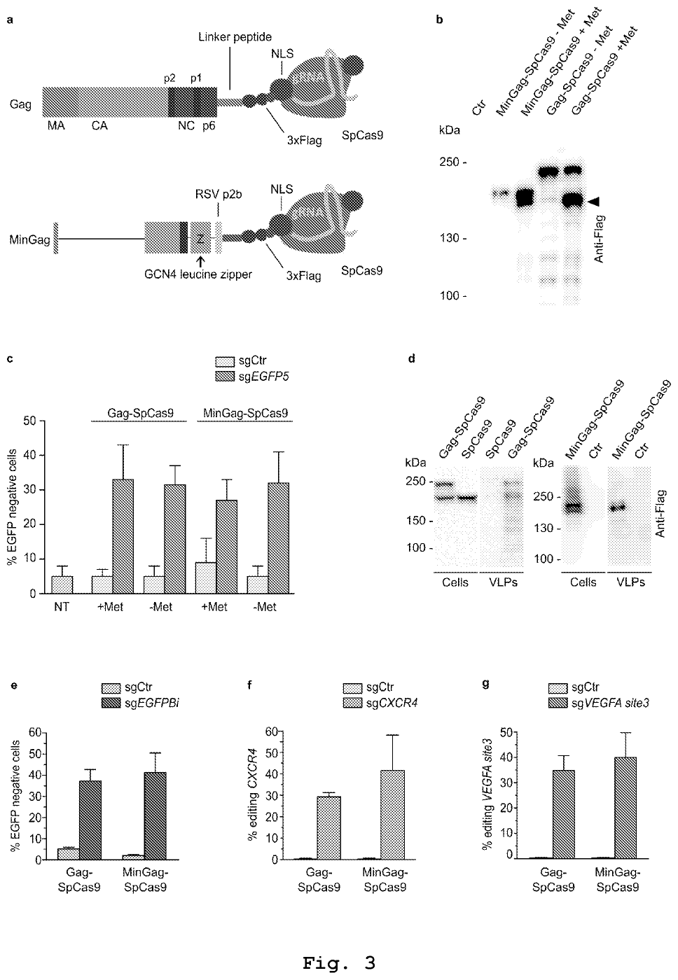

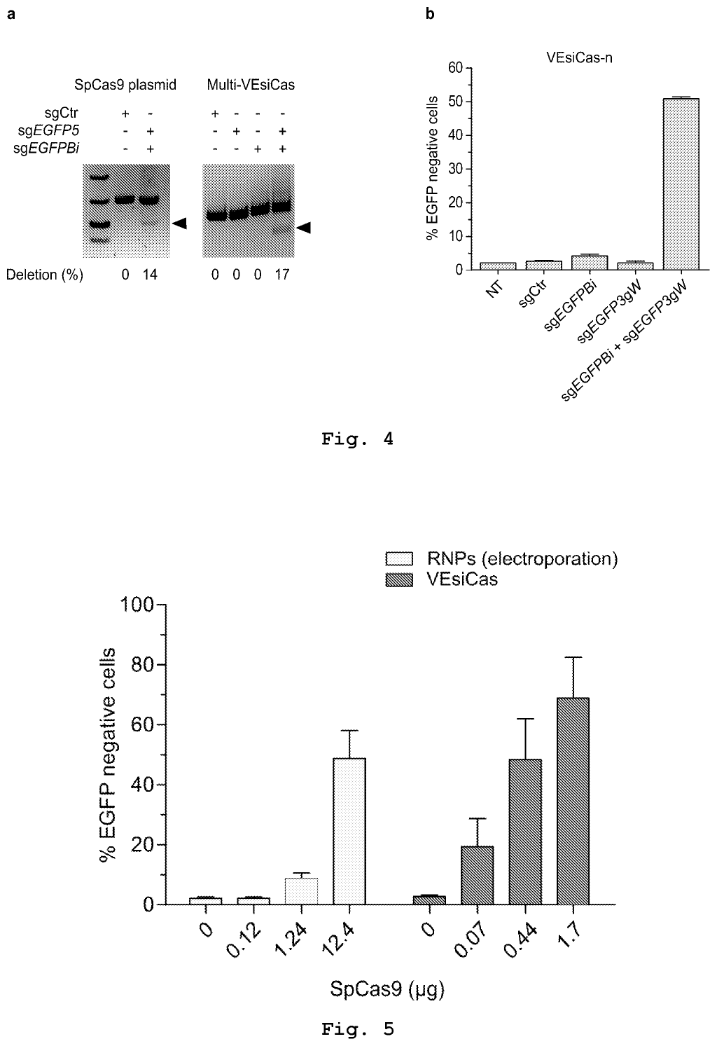

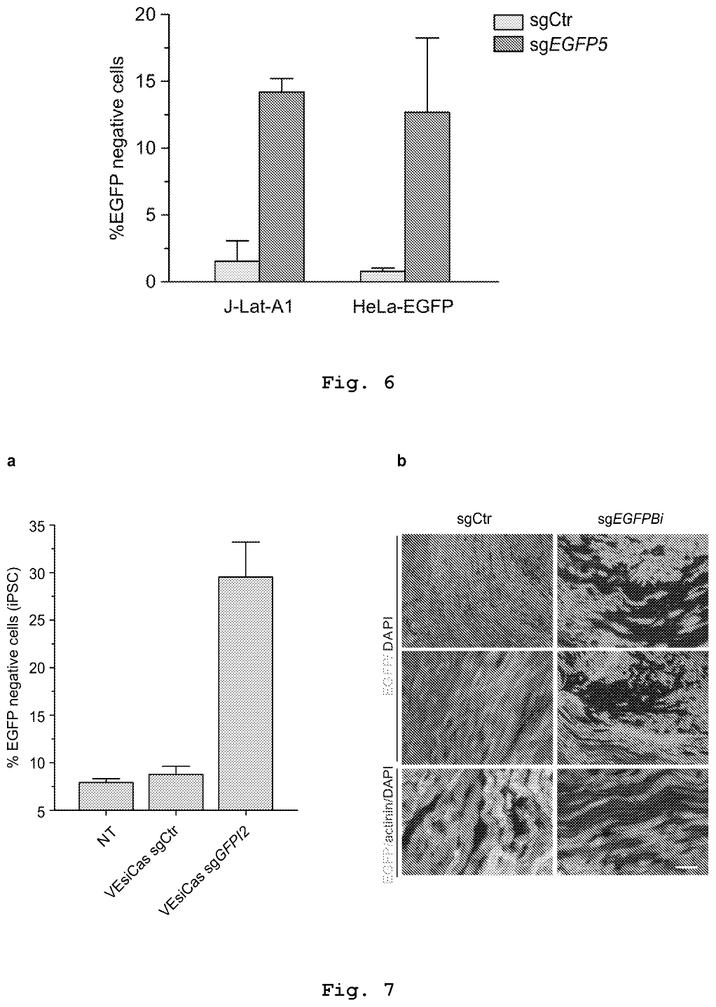

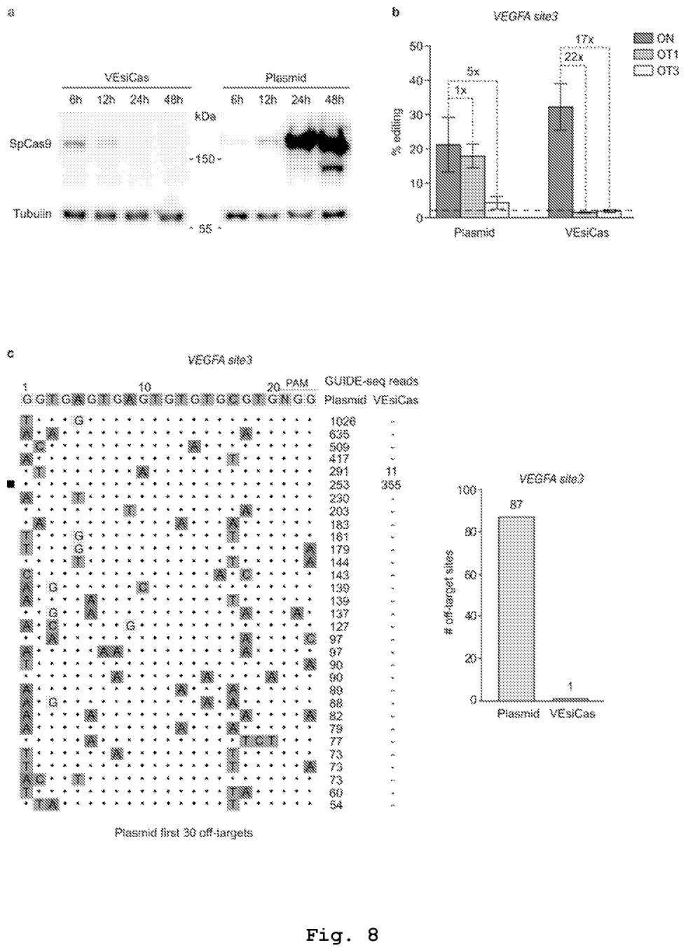

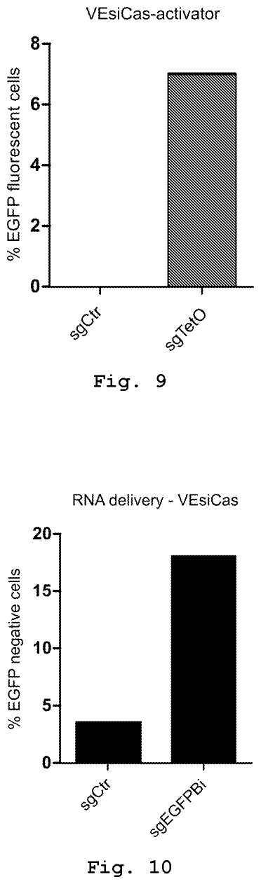

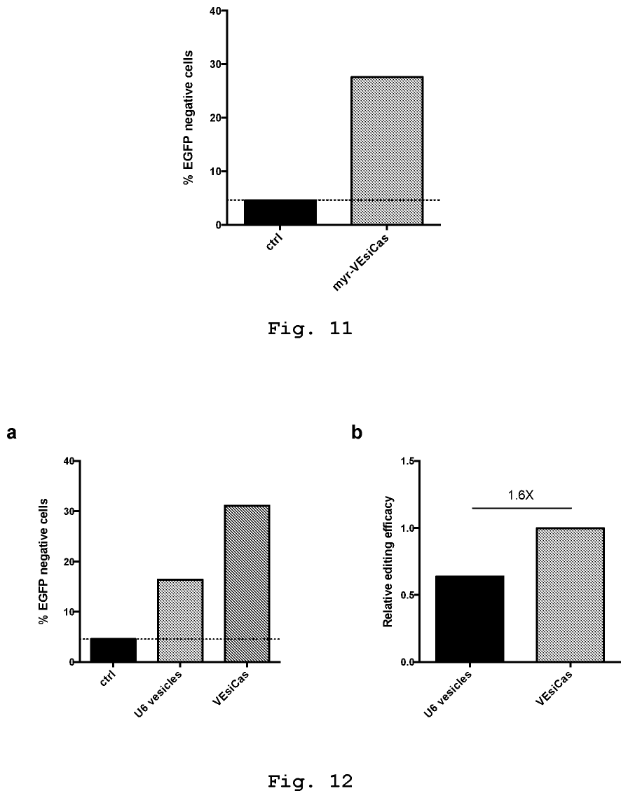

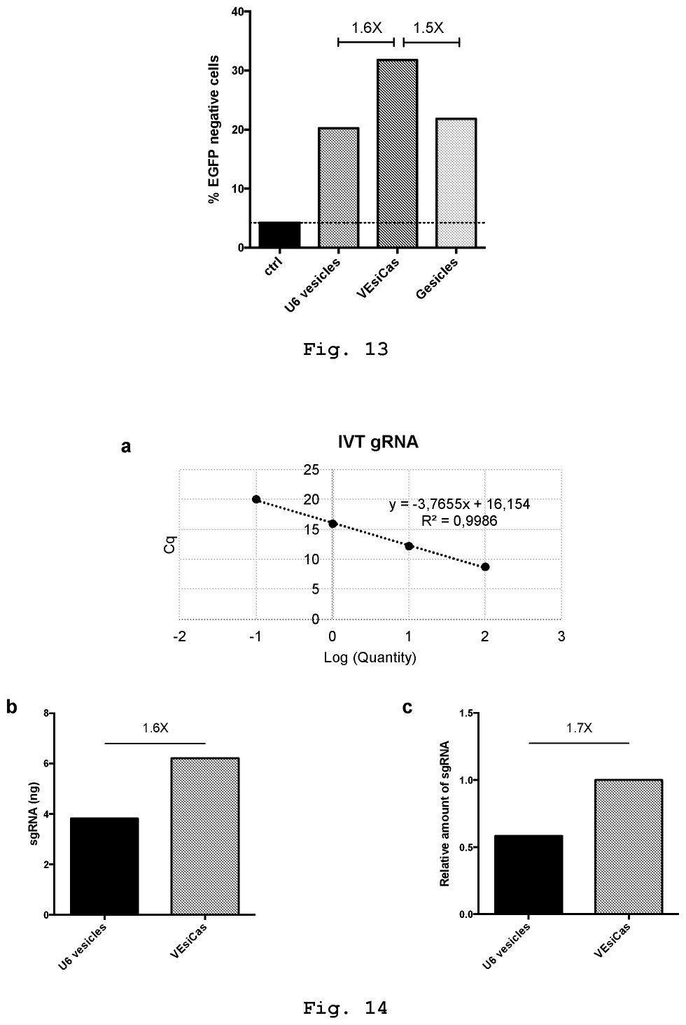

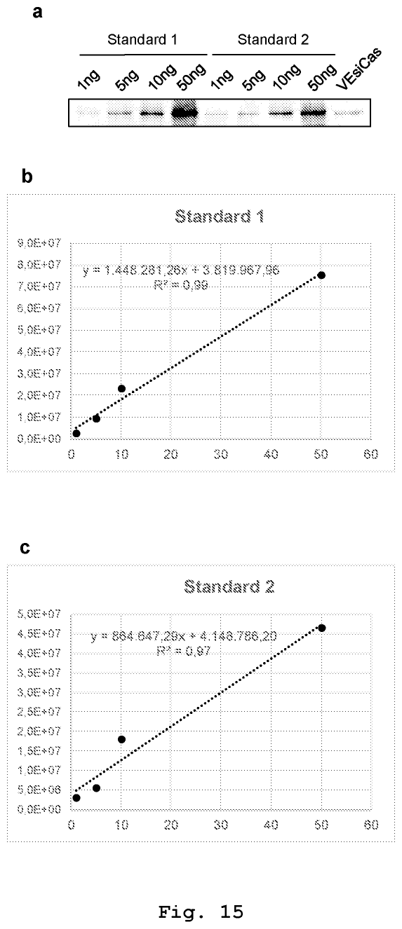

VSV-G induced vesicles were reported to mediate protein transfer in the absence of additional viral components (Mangeot et al. 2011). We tested whether VSV-G vesicles could be adapted to DNA-free delivery of SpCas9 and sgRNA. SpCas9 and sgRNA towards the EGFP coding sequence (sgEGFP5) were expressed together with VSV-G in HEK293T cells and the derived conditioned clarified medium was applied onto a fluorescent reporter cell line, HEK293-EGFP. The expression of EGFP was poorly altered in these conditions indicating inefficient genome editing, while efficient editing was observed by transfecting SpCas9 together with the sgRNA (FIG. 1a). The limited editing activity was not explained by the lack of delivered protein since both vesicles and target cells were positive for SpCas9 (FIG. 2a). These results prompted us to evaluate whether the limiting factor for vesicles activity could be the insufficient amounts of incorporated sgRNA. To this aim the HEK293-EGFP target cells were transfected with plasmids expressing sgEGFP5 before treatment with VSV-G vesicles carrying SpCas9. In these experimental conditions, we obtained editing levels that were closer to those observed in cells transfected with SpCas9 and the sgRNA (FIG. 1a, compare sixth and second column of the graph). These results clearly suggested that the limited editing observed with the SpCas9/VSV-G preparations was due to the inefficient delivery of the sgRNA. We speculated that poor sgRNA delivery could be due to inefficient formation of SpCas9-sgRNA RNP complexes during vesicle production. In particular, the Pol III-synthesized sgRNAs in the nuclei may have poorly coupled with cytoplasmic SpCas9 to form RNPs at cell periphery, close to the nascent VSV-G vesicles. To test this hypothesis we employed a T7 RNA polymerase-driven transcription system (Buchholz et al. 1999; Habjan et al. 2008) which catalyzes RNA synthesis in the cytoplasm (schematized in FIG. 1b). The sgRNAs were cloned downstream the T7 promoter and the 5′ HDV ribozyme was introduced between the sgRNA coding sequence and the T7 RNA polymerase terminator to induce the formation of mature sgRNAs with unmodified 3′ constant regions (Nissim et al. 2014). The VSV-G enveloped SpCas9 vesicles were produced in cell lines stably expressing the T7 RNA polymerase and resistant to the toxicity induced by high levels of uncapped 5′-triphosphate cytoplasmic RNA, generated by this transcriptional system (Habjan et al. 2008; D.-H. Kim et al. 2004; Pichlmair et al. 2006) (FIG. 2b). The derived VSV-G Enveloped SpCas9 Vesicles, VEsiCas, produced in BSR-T7/5 cells expressing sgEGFP5, were verified for SpCas9 incorporation (FIG. 2c). These VEsiCas induced at least 50% loss of EGFP fluorescence in HEK293-EGFP cells, thus very similar to knockouts observed with VSV-G/SpCas9 vesicle treatments of cells pre-transfected with sgEGFP5 (FIG. 1c). VEsiCas were then tested towards two genomic loci, CXCR4 and the VEGFA, commonly used as a benchmark in genome editing experiments involving Cas9, generating indels in both loci (FIG. 1d,e).

SpCas9 pseudotransduction was also tested using lentiviral-based viral-like particles (lenti-VLPs). The HIV-1 Gag domain or a reduced portion of it (MinimalGag) were reported to generate viral-like particles (VLP), described to efficiently transfer protein cargoes to recipient cells (Accola et al. 2000). SpCas9 fused to Gag or MinimalGag was functionally active in genome editing activity against the EGFP locus (FIG. 3a-c). The two SpCas9 chimeras were used to produce lentiviral-based VLPs (lenti-VLPs) in BSR-T7/5 cells, which produced high percentages of indels in the EGFP, CXCR4 and VEGFA loci (FIG. 3d-g). However, since no dramatic improvements in genome editing were obtained with lenti-VLPs, the VEsiCas carrying exclusively the VSV-G viral element were used hereafter.

Overall our data clearly showed that VEsiCas efficiently deliver SpCas9-sgRNA RNPs free from encoding DNA or additional elements of viral origin. A key factor to obtain highly efficient genome editing particles was the relocation of sgRNA expression from the nucleus to the cytoplasm of producing cells, which was obtained in appropriate permissive cells through a cytoplasmic T7 RNA polymerase.

Multiplexed VEsiCas

Since genome editing applications such as targeted genomic deletions may require the simultaneous delivery of more than one sgRNA, we evaluated the possibility to incorporate multiple guides into VEsiCas (Multi-VEsiCas). Multi-VEsiCas were produced in BSR-T7/5 cells expressing two T7 driven EGFP-targeting sgRNAs (sgEGFP5 and sgEGFPBi). Incubation of HEK293-EGFP reporter cells with Multi-VEsiCas carrying both targeting sgRNAs generated the expected deletion in the EGFP locus with ˜17% efficiency, which was similar to the one obtained with transient transfection of plasmids encoding SpCas9 and the corresponding sgRNAs (˜14%) (FIG. 4a). Conversely, VEsiCas carrying each individual sgRNA (either sgEGFP5 or sgEGFPBi) were not able to produce any detectable deletion into the target locus (FIG. 4a). The flexibility of the VEsiCas delivery platform was further demonstrated by incorporating the SpCas9 nickase (SpCas9-n, D10A mutant) (Ran et al. 2013) to generate VEsiCas-n carrying two closely positioned guides targeting the EGFP coding sequence. VEsiCas-n were prepared either with individual sgRNAs (sgEGFPBi or sgEGFP3gW) or with the sgRNAs in combination (sgEGFPBi and sgEGFP3gW). Treatment of HEK293-EGFP cells with VEsiCas-n carrying both sgRNAs produced a robust decrease in the number of fluorescent cells (50%), whereas VEsiCas-n delivering individual sgRNAs did not downregulate EGFP expression, as expected (FIG. 4b). These data indicate that more than one sgRNA can be simultaneously delivered by VEsiCas, thus demonstrating their applicability to generate deletions and to deliver SpCas9 nickase.

VEsiCas Editing Efficiency in Cells and In Vivo

Increasing amounts of VEsiCas resulted into a proportional increase in the editing activity (FIG. 5). Moreover, side-by-side comparison with electroporation protocols, which are often used to deliver Cas9 into cells for genome editing, revealed that VEsiCas are more efficient. In fact, compared to electroporation, a lesser amount of SpCas9 delivered via VEsiCas was required to obtain similar percentages of EGFP knock-out cells (FIG. 5). The efficacy of VEsiCas was tested in different cell lines, including adherent cells (HeLa), suspension cells (J-Lat-A1) and in human induced pluripotent stem cells (iPSCs) expressing EGFP, showing a robust decrease in the number of fluorescent cells in all the tested models (FIG. 6 and FIG. 7a). Finally, VEsiCas were tested in the cardiac muscle of EGFP transgenic mice. VEsiCas were injected in the heart of 5 days-old mice, which were analyzed for levels of EGFP fluorescence 10 days after treatment. The cardiac tissue from treated animals was stained using antibodies against α-actinin to specifically evaluate EGFP fluorescence in cardiomyocytes. As shown in FIG. 7b, large areas of non-fluorescent cardiomyocytes could be observed close to the VEsiCas injection site (around 30% of EGFP-negative cells).

These data show that VEsiCas are efficient tools to deliver SpCas9 RNPs for genome editing in culture cells as well as in vivo.

Limited Off-Target Activity by SpCas9 Delivered Through VEsiCas

The off-target activity produced by Cas9 is still one of the main limitations for its therapeutic use. The transient expression of the nuclease in target cells has been shown to limit non-specific cleavages (Petris et al. 2017; S. Kim et al. 2014). To address this point, the kinetic of SpCas9 intracellular levels delivered through VEsiCas in comparison with the amounts of nuclease expressed by transfected plasmid was examined. SpCas9 from VEsiCas was detected in target cells 6 hours post transduction, and gradually disappeared within the following 18 hours (FIG. 8a). Conversely, the nuclease intracellular levels produced by an expression plasmid were clearly detected 12 hours post transfection and increasingly accumulated thereafter (FIG. 8a). To evaluate the extent of the off-target activity generated by SpCas9 delivered through VEsiCas or by transfection, indels formation was measured at two previously validated off-target sites associated with the editing of the VEGFA locus (VEGFA OT1 and OT3) (Petris et al. 2017). The Tracking of Indels by DEcomposition (TIDE) analysis (Brinkman et al. 2014) revealed that SpCas9 delivered through VEsiCas produced higher levels of on-target activity than transfected SpCas9, while causing background levels of indels at both off-target sites (FIG. 8b). Strikingly, VEsiCas produced at least 17- and 22-fold less indels at the OT1 and OT3, respectively, relative to the on-target cleavage, while transfected SpCas9 cleaved OT1 similarly to the on-target site, and OT3 was edited just 5 times less than the intended target. A deeper off-target analysis on the VEGFA locus was performed through GUIDE-seq (Tsai et al. 2014), a genome-wide approach (FIG. 8c). The analysis revealed that exclusively one off-target site with hundreds less GUIDE-seq reads than the on-target could be found in VEsiCas transduced cells (355 reads on-target and 11 reads off-target). In contrast, SpCas9 expressed from a transfected plasmid cleaved with similar efficiency this on- and the associated off-target site (253 on-target and 291 off-target GUIDE-seq reads). Moreover, SpCas9 expressed from a transfected plasmid generated four additional off-target sites characterized by a higher number of GUIDE-seq reads (from 417 to 1026) than those obtained with the on-target.

In conclusion, the off-target analysis performed on VEGFA locus, a gold-standard for the evaluation of SpCas9 specificity, revealed that the VEsiCas produced a more specific genomic modification, which correlates with the quick clearance of the nuclease from the target cells.

VEsiCas Activators of Gene Expression.

Traceless regulation of gene expression could be desirable for several aims from in vitro basic research to in vivo disease treatment. Several different fusions of RNA guided nucleases, in particular dead SpCas9 (dSpCas9), with transcriptional activators, repressor or chromatin modifiers (e.g. VP64, VPR, KRAB, p300) were employed to artificially control gene expression (Vora et al. 2016; Y. Gao et al. 2016). To demonstrate the feasibility of the inclusion of such technologies into VEsiCas, the particles were purified from supernatants of BSR-T7/5 cells transfected with plasmid expressing dSpCas9-VP64 transcriptional activator and sgRNAs. Before transduction with VEsiCas-activator, target cells were transfected with a model plasmid encoding EGFP gene under control of a minimal CMV promoter, which express EGFP only if additional transcription factors bind the promoter of nearby sequences. As shown in FIG. 9, VEsiCas-activator containing dSpCas9-VP64 and sgRNAs targeting nearby the promoter sequence (sgTetO) upregulated EGFP expression from the reporter model in 7% of the cells, while no EGFP positive cells were observed after treatment with non-targeting (sgCtr) VEsiCas activators. This data clearly indicates the possibility of deliver by VEsiCas of RNPs formed by dSpCas9 fusion proteins and sgRNAs for purposes of gene regulation.

VEsiCas Delivery of RNA.

RNA highly expressed in the cytoplasm by T7 RNA polymerase should be capable of packaging into VEsiCas. Indeed, the presence of a Cas9 molecule was not required for sgRNA delivery by VEsiCas. In FIG. 10 EGFP positive target cells were pre-transfected with SpCas9 plasmid and subsequently transduced with VEsiCas collected from producing cells expressing sgRNA, but not SpCas9. As expected SpCas9 transfected cells if transduced with targeting sgRNA (sgEGFPBi) had a robust increased in the number of EGFP negative cells (18%) due to genome editing, while near background levels of non-fluorescent cells (4%) were observed in control treated samples (sgCtr). These data indicate that RNAs, and in particular sgRNA, can be packaged and delivered by VEsiCas independently by SpCas9 presence and have the desired activity into target cells.

Editing Activity of VEsiCas Incorporating Myristylated SpCas9

To further increase nuclease incorporation by VEsiCas, we evaluated the possibility to induce SpCas9 binding to the plasma membrane through myristylation of the protein. To this aim we fused the myristylation signal of HIV-1 matrix (Lee and Linial, J. Virol. 1994 Oct; 68(10): 6644-6654; Uniprot KB ID P04591) to the N-terminus of SpCas9 and used this construct to produce VEsiCas (myr-VEsiCas). We then measured the editing efficiency by targeting the EGFP locus in HEK293-GFP, obtaining 27.6% of EGFP-negative cells (FIG. 11). These data demonstrate that SpCas9 fused to an N-terminal myristylation signal is catalytically active, is correctly incorporated in VEsiCas and is able to induce genomic modifications in the target cell.

Materials and Methods

Plasmids and oligonucleotides. The pUC19 sgRNAs expression plasmids transcribing sgRNA form U6 promoter and used for the initial experiments on VSV-G vesicles production were previously published (Petris et al. 2017). The Gag-SpCas9 (SEQ ID NO.: 47) was obtained through the fusion of the Gag coding sequence with 3×FLAG-SpCas9 encoded by the previously published pX-SpCas9 plasmid (Petris et al. 2017). dSpCas9-VP64 was expressed from the previously published pcDNA-dSpCas9-VP64 (Perez-Pinera et al. 2013). pTRE-EGFP was obtained by cloning a fragment NotI and EcoRI from pEGFP-N1 (Clontech) into pTRE-tight (Clontech).

MinimalGag-SpCas9 (SEQ ID NO.: 48) was assembled in pCDNA3 by subcloning the SpCas9 coding sequence from pX-SpCas9 and MinimalGag from A-Zwt-p2b previously published plasmid (Accola et al. 2000). Afterwards an additional version of both construct was obtained by removing through site directed mutagenesis a methionine (in position 517 of SEQ ID NO.: 49 and in position 164 of SEQ ID NO.: 50) in the linker peptide derived from pX-Cas9 that lead to unfused SpCas9 production (FIG. 3).

For VLPs and VEsiCas production in BSR-T7/5 cells sgRNAs were transcribed from a pVAX-T7-sgRNA expression plasmid having a T7 promoter driven cassette, cloned into the pVAX plasmid (Thermo Fisher Scientific) using the NdeI and XbaI sites. SEQ ID NO.: 51 describes the transcription unit inserted in the pVAX plasmid using the NdeI and XbaI sites to obtain the pVAX-T7-sgRNA plasmid. sgRNA oligos were cloned in pVAX-T7-sgRNA using a double BsmBI site inserted before the sgRNA constant portion (the list of oligonucleotides used to clone sgRNAs is provided in Table 1).

TABLE 1
Sequences of oligonucleotides used to
construct pVAX-T7-sgRNA expression plasmids and
sequences of relative target sites

| sgRNA | oligo 1 (*) | oligo 2 (*) | target site (**) |
|---|---|---|---|
| EGFP5 | tataGGAAGTTCGAGG | aaacGGGTGTCGCCCTCG | ggtGAAGTTCGAGGGCGACACCCTGGtga |
| | GCGACACCC | AACTTCCCC | SEQ ID NO.: 13 |
| | SEQ ID NO.: 1 | SEQ ID NO.: 2 | |
| EGFPBi | tataGGGCACGGGCAG | aaacCCGGCAAGCTGCCC | ccaCCGGCAAGCTGCCCGTGCCCTGGccc |
| | CTTGCCGG | GTGCCCCC | SEQ ID NO.: 14 |
| | SEQ ID NO.: 3 | SEQ ID NO.: 4 | |
| EGFP3gW | tataGGGCTCGTGACC | aaacTAGGTCAGGGTGGT | accCTCGTGACCACCCTGACCTACGGcgt |
| | ACCCTGACCTA | CACGAGCCCCC | SEQ ID NO.: 15 |
| | SEQ ID NO.: 5 | SEQ ID NO.: 6 | |
| GFPI2 | tataGGTGGGCACCGG | aaacCGGGGAAGCCGGTG | ggtGGTGGGCACCGGCTTCCCCGAGGaca |
| | CTTCCCCG | CCCACCCCC | SEQ ID NO.: 16 |
| | SEQ ID NO.: 7 | SEQ ID NO.: 8 | |
| VEGFA | tataGGTGAGTGAGTG | aaacCACGCACACACTCA | gtgGGTGAGTGAGTGTGTGCGTGTGGggt |
| site3 | TGTGCGTG | CTCACC | SEQ ID NO.: 17 |
| | SEQ ID NO.: 9 | SEQ ID NO.: 10 | |
| VEGFA | — | — | gtgAGTGAGTGAGTGTGTGTGTGGGGggg |
| OT1 | | | SEQ ID NO.: 18 |
| VEGFA | — | — | atgTGTGGGTGAGTGTGTGCGTGAGGaca |
| OT3 | | | SEQ ID NO.: 19 |
| CXCR4 | tataGGAAGCGTGATG | aaacCCTCTTTGTCATCA | aagGGAAGCGTGATGACAAAGAGGagg |
| | ACAAAGAGG | CGCTTCCCC | SEQ ID NO.: 20 |
| | SEQ ID NO.: 11 | SEQ ID NO.: 12 | |
| TetO | tataGGTACGTTCTCT | aaacTATCAGTGATAGAG | acaTACGTTCTCTATCACTGATAGGGagt |
| | ATCACTGATA | AACGTACC | SEQ ID NO.: 37 |
| | SEQ ID NO.: 35 | SEQ ID NO.: 36 | |
| Ctr | tataGGAGACGGATTG | aaacAGAGACGTCAATCC | — |
| | ACGTCTCT | GTCTCC | |
| | SEQ ID NO.: 38 | SEQ ID NO.: 39 | |

(*) Lowercase indicates sticky ends used for cloning into pVAX-T7-sgRNA plasmid.
(**) Mismatches and PAM are in bold. Context sequence around target site are in
lowercase.

pVAX-T7-sgRNA included a 5′ HDV ribozyme (nucleotides 132 to 219 of SEQ ID NO.: 51) (Nissim et al. 2014) designed to cleave the 3′-end of the sgRNA containing the T7 terminator (nucleotides 266 to 364 of SEQ ID NO.: 51) and the ribozyme itself. The Cas9-Nickase construct was obtained by inserting D10A mutation in the SpCas9 coding sequence (UniProtKB ID Q99ZW2). The T7 RNA polymerase coding sequence (SEQ ID NO.: 40) was amplified using primers T7 fw—HindIII (SEQ ID NO.: 21) and T7 rev—XbaI (SEQ ID NO.: 22), from the genome of BSR-T7/5 cells and cloned in place of EGFP into the pEGFP-N1 plasmid (Life technologies) using the Hindlll/XbaI sites to obtain the pKANA-T7-RNA-Pol plasmid. Plasmids were verified by Sanger sequencing.

Cell cultures and transfection. BHK21-derived producing cells stably expressing T7 polymerase (BSR-T7/5) as disclosed in (Buchholz et al. 1999). To select for cells that retain the T7 RNA polymerase construct, every other passage the media was supplemented with 1 μg/mL G418 (Gibco-Life technologies). HEK293T cells were obtained from ATCC. HEK293-EGFP cells were generated by stable transfection of pEGFP-IRES-Puromicin plasmid (Petris et al. 2017) and selected with 1 μg/ml of puromicin. BHK-21 (ATCC CCL-10), Vero (ATCC-CCL-81), HeLa (ATCC-CCL-2) and all the cell lines described above were cultured in DMEM supplemented with 10% heat inactivated FBS, 2 mM L-glutamine, 10 U/ml penicillin, and 10 μg/ml streptomycin. J-Lat-A1 are Jurkat cells (Jordan et al. 2003) which had been latently transduced by a HIV-1 vector encoding EGFP. J-Lat-A1 were cultured in RPMI medium, supplemented with 10% FBS and pen/strep antibiotics. EGFP expression was induced with 10 nM TPA (12-O-tetradecanoylphorbol-13-acetate) treatment for 24 hours. To obtain HeLa-EGFP, HeLa cells were transfected with the pEGFP-C1 plasmid (Clontech) using FuGENE HD Transfection reagent (Promega). After selection in culture medium with 400 μg/mL G418 (Life Technologies) for approximately 10 days, cells expressing high levels of EGFP were enriched by FACS sorting and propagated as a polyclonal cell population. Stock cultures of HeLa-EGFP cells (˜95% EGFP-positive cells) were maintained in culture medium supplemented with 200 μg/mL G418. Transgenic human iPSCs constitutively expressing GFP were derived from a commercial Human Episomal iPSC Line (Gibco, Thermo Fisher Scientific), originally derived from CD34+ cord blood using an EBNA-based episomal system. Human iPSC clones stably expressing copGFP under control of the ubiquitous cytomegalovirus promoter were generated by infection with the pGZ-CMV-copGFP lentiviral vector (System Biosciences). Zeocin-based clone selection was started 72 hours post infection for days. Resistant colonies were manually picked, expanded clonally and characterized for their pluripotency competence. Human iPSC lines were grown on feeder-free Geltrex-coated dishes, cultured in StemMACS iPS-Brew XF medium (Miltenyi Biotech). All cell lines were verified Mycoplasma-free (PlasmoTest, Invivogen).

Transfection experiments were performed in 12-24 multi-wells plates with 250-1000 ng of each plasmid using the TranslT-LT1 (Mirus) reagent, according to manufacturer's instructions. Cells were collected 2-4 days after transfection or as described.

VSV-G/SpCas9 vesicles, lenti-VLPs and VEsiCas production. For VSV-G/Cas9 vesicles production, a confluent 100 mm dish of HEK293T cells was transfected with 15 μg of pxCas9, Gag-SpCas9, or pCDNA3-MinGag-SpCas9, 15 μg of the desired pUC-U6-sgRNA and 3 μg of VSV-G plasmids using the polyethylenimine (PEI) method (Casini et al. 2015). Subsequent productions were performed in BSR-T7/5 cells using the conditions reported above except for the pVAX-T7-sgRNA plasmid that substituted pUC-U6-sgRNA for the RNA guide expression. For Multi-VEsiCas during deletion experiments 7.5 μg of each pVAX-T7-sgRNA (sgEGFP5 and sgEGFPBi) targeting EGFP were used. For VEsiCas-n production, 15 μg of Cas9-Nickase plasmid and 7.5 μg of each pVAX-T7-sgEGFPBi and pVAX-T7-sgEGFP3gW were used. For VEsiCas transcription regulators BSR-T7/5 cells were transfected with 15 μg of pcDNA-dSpCas9-VP64 and 15 μg of control (sgCtr) or promoter targeting (sgTetO) pVAX-T7 plasmid expressing sgRNAs. For VEsiCas delivering RNA molecules without SpCas9 cells where transfected only with 15 μg of pVAX plasmid expressing the indicated sgRNA. After 12 hours of incubation, the medium was replaced with fresh complete DMEM and 48 hours later the supernatant was collected, centrifuged at 400×g for 5 minutes and filtered through a 0.22 μm PES filter. VLPs and VEsiCas were then concentrated and purified through a 20% sucrose cushion by ultracentrifugation for 2 hours at 150000×g (4° C.) and resuspended in suitable volumes of complete medium (DMEM, RPMI or StemMACS iPS-Brew XF medium, according to the target cells) or 1×PBS for mice injections and stored at −80° C. The amount of SpCas9 or Gag-SpCas9 and MinGag-SpCas9 chimeras produced into the VSV-G vesicles was evaluated through Western blot analysis using purified SpCas9 as a standard. Unless indicated, for each single transduction experiment were used vesicles containing about 1 μg of SpCas9. The reference recombinant SpCas9 protein was produced in bacteria (see below) according to (Gagnon et al. 2014) and quantified through Coomassie staining. The efficiency of SpCas9 incorporation into VEsiCas was obtained by calculating the amount of incorporated SpCas9 measured through Western blot using a recombinant protein of known concentration as a standard over the total amount of proteins in the VEsiCas preparation quantified by Bradford assay (Bio-Rad). The efficiency of gRNA incorporation was evaluated by standard qRT-PCR procedures using the forward primer 5′-TAAGAGCTATGCTGGAAACAGC-3′ (SEQ ID No. 52) (gRNA for) and the reverse primer 5′-GACTCGGTGCCACTTTTTCA-3′ (SEQ ID No. 53) (gRNA rev) for the optimized sgRNA scaffold and the forward primer 5′-AGCTAGAAATAGCAAGTTAAAATAAGG-3′ (SEQ ID No. 54) (gRNAopt for) and the reverse primer 5′-GACTCGGTGCCACTTTTTCA-3′ (SEQ ID No. 55) (gRNAopt rev) for the standard sgRNA scaffold (Zhang et al. 2016). Note that the reverse primer is common for both sgRNA configurations.

Delivery in target cells. The day before transduction, 1×10⁵ HEK293T, HEK293-EGFP, HeLa-EGFP or J-LAT-A1 were seeded in a 24-wells plate. VEsiCas and VLPs were delivered into target cells by spinoculation for 2 hours at 1600×g at 20° C. (30 min at 1000×g and 24° C. for Human iPSCs), followed by an overnight incubation at 37° C. J-Lat-A1 cells were induced by TPA (Sigma-Aldrich) and analyzed for genome editing three days after the last transduction or for EGFP reduction at least seven days after the treatment. For VEGFA and CXCR4 loci triple transduction experiments were performed. Cells were trypsinized 24 hours after each transduction; ⅔ of cells were collected for genomic analysis and ⅓ was subcultured for following treatments, one each 48 hours. Cells were collected for final analysis three days after the last treatment.

SpCas9-sgRNA electroporation. Recombinant SpCas9 protein (UniProtKB ID Q99ZW2) was produced in bacteria according to (Gagnon et al. 2014). Briefly, the pET-28b-Cas9-His plasmid (Addgene #47327) was used to express SpCas9 in E. coli Rosetta cells (Novagen), which were grown for 12 hours at 37° C., followed by 24 hours induction at 18° C. Purification was performed on his-tag resin (G-Biosciences), column was washed with mM Tris pH 8, 30 mM Imidazole, 500 mM NaCl and eluted with 20 mM Tris pH 8, 500 mM Imidazole, 500 mM NaCl. After dialyses into 20 mM Tris, 200 mM KCl, 10 mM MgCl₂ single-use aliquots were stored at −80° C. To produce in vitro transcribed sgRNAs we first PCR amplified the pVAX-T7-sgEGFPBi and pVAX-T7-sgCtr with primers T7 promoter fw and gRNA end rev (Table 2).

TABLE 2
Sequences of the oligonucleotides
used for PCR amplifications.

| oligo | sequence |
|---|---|
| T7 fw-HindIII | ACTAAGCTTGTCGACCATGAACACGATT |
| | AACATCG |
| | SEQ ID NO.: 21 |
| T7 rev-XbaI | TAATCTAGATTACGCGAACGCGAAGTC |
| | SEQ ID NO.: 22 |
| T7 promoter fw | GAAATTAATACGACTCACTATAGG |
| | SEQ ID NO.: 23 |
| gRNA end rev | AAGCACCGACTCGGTGCCA |
| | SEQ ID NO.: 24 |
| EGFP fw | ACCATGGTGAGCAAGGGCGAGGA |
| | SEQ ID NO.: 25 |
| EGFP rev | AGCTCGTCCATGCCGAGAGTGATC |
| | SEQ ID NO.: 26 |
| VEGFA site3 ON fw | GCATACGTGGGCTCCAACAGGT |
| | SEQ ID NO.: 27 |
| VEGFA site3 ON rev | CCGCAATGAAGGGGAAGCTCGA |
| | SEQ ID NO.: 28 |
| VEGFA site3 OT1 fw | CAGGCGCCTTGGGCTCCGTCA |
| | SEQ ID NO.: 29 |
| VEGFA site3 OT1 rev | CCCCAGGATCCGCGGGTCAC |
| | SEQ ID NO.: 30 |
| VEGFA site3 OT3 fw | AGTCAGCCCTCTGTATCCCTGGA |
| | SEQ ID NO.: 31 |
| VEGFA site3 OT3 rev | GAGATATCTGCACCCTCATGTTCAC |
| | SEQ ID NO.: 32 |
| CXCR4 fw | AGAGGAGTTAGCCAAGATGTGACTTTGA |
| | AACC |
| | SEQ ID NO.: 33 |
| CXCR4 rev | GGACAGGATGACAATACCAGGCAGGATA |
| | AGGCC |
| | SEQ ID NO.: 34 |

This PCR product, containing the T7-promoter and the complete sequence of the sgRNA, was used for in vitro transcription using HiScribem T7 High Yield RNA Synthesis Kit (New England Biolabs) following manufacturer's instructions. TRIzol (Invitrogen) purified sgRNAs, precipitated with isopropanol and washed with 75% ethanol, were analyzed by acrylamide gel electrophoresis and quantified using a NanoDrop spectrophotometer (Thermo Fisher Scientific).

Purified sgRNAs were mixed with recombinant SpCas9 immediately before electroporation by incubating 12.4 μg of SpCas9 with 3.1 μg of sgRNA (or as indicated with a 4:1 mass ratio between protein and RNA) in 20 mM Hepes (pH 7.5), 150 mM KCl, 1 mM MgCl₂, 10% (vol/vol) glycerol, and 1 mM DTT at 37° C. for 10 min. 2.5×10⁵ HEK293-EGFP cells were nucleofected using the Q001 protocol in 120 mM K₂HPO₄/KH₂PO₄ pH 7.2, 15 mM MgCl₂, 10 mM glucose, 5 mM KCl, using Lonza Nucleofector II. Cells were analyzed for EGFP loss seven days after electroporation.

Detection of SpCas9-induced mutations. EGFP expression was analyzed by Invitrogenm Tali™ Image-based Cytometer. In the comparative analysis with electroporated RNPs, the analysis of EGFP expressing cells was performed by FACS (FACSCanto, BD Biosciences). In order to detect indels in the CXCR4 and VEGFA loci, genomic DNA was isolated using the DNeasy® Blood & Tissue kit (Qiagen). PCRs on purified genomic DNA were performed using the Phusion High-Fidelity DNA polymerase (Thermo Fisher Scientific). Samples were amplified using the oligonucleotides listed in Table 2. Purified PCR products were analysed by sequencing and applying the TIDE tool³⁶. Detection of the deletion in the EGFP locus after Multi-VEsiCas treatment was revealed by PCR amplification using oligonucleotides EGFP fw and EGFP rev.

GUIDE-seq. 2×10⁵ HEK293T cells were transfected with 750 ng of Cas9 expressing plasmid, together with 250 ng of VEGFA site3 sgRNA-coding plasmid or an empty pUC19 plasmid (both described in (Petris et al. 2017)), 10 pmol of the bait dsODN containing phosphorothioate bonds at both ends (designed identical to the GUIDE-seq protocol) (Tsai et al. 2014) and 50 ng of a pEGFP-IRES-Puro plasmid (Petris et al. 2017), expressing both EGFP and the puromycin resistance gene. VEsiCas targeting VEGFA site3 were delivered by spinoculation 6 hours following transfection of dsODN and EGFP-IRES-Puro coding plasmids. The following day, cells were trypsinised and replated. The procedure was repeated each 48 h. After the last treatment, cells were detached and selected with 2 μg/ml of puromycin for 48 hours. Cells were then collected and genomic DNA was extracted using the DNeasy Blood and Tissue kit (Qiagen) following the manufacturer's instructions and sheared to an average length of 500 bp with the Bioruptor Pico sonication device (Diagenode). Library preparations were performed with the adapters and primers according to previous work (Tsai et al. 2014). Libraries were quantified with the Qubit dsDNA High Sensitivity Assay kit (Invitrogen) and sequenced with the MiSeq sequencing system (Illumina) using an Illumina Miseq Reagent kit V2-300 cycles (2×150 bp paired-end). Raw sequencing data (FASTQ files) were analyzed using the GUIDE-seq computational pipeline (Tsai et al. 2016). After demultiplexing, putative PCR duplicates were consolidated into single reads. Consolidated reads were mapped to the human reference genome GrCh37 using BWA-MEM37; reads with mapping quality lower than 50 were filtered out. Upon the identification of the genomic regions integrating double-stranded oligodeoxynucleotide (dsODNs) in aligned data, off-target sites were retained if at most eight mismatches against the target were present and if absent in the background controls. Visualization of aligned off-target sites is available as a color-coded sequence grid.

Western blots. Collected cells or supernatants containing VSV-G vesicles were lysed in NEHN buffer (20 mM HEPES pH 7.5, 300 mM NaCl, 0.5% NP40, NaCl, 1 mM EDTA, 20% glycerol) supplemented with 1% of protease inhibitor cocktail (Pierce). Protein extracts were separated by SDS-PAGE using the PageRuler Plus Protein Standards as the standard molecular mass markers (Thermo Fisher Scientific). After electrophoresis, samples were transferred onto 0.22 μm PVDF membranes (GE Healthcare). The membranes were incubated with mouse anti-FLAG (Sigma) for detecting Gag-Cas9, MinGag-Cas9 and SpCas9, mouse anti-α-tubulin (Sigma) and with the appropriate HRP conjugated goat anti-mouse (KPL) secondary antibody for ECL detection. The Guide-It™ Cas9 Monoclonal Antibody (Clone TG8C1—Clontech) was used to quantify SpCas9 by western blotting using recombinant SpCas9 as reference. Images were acquired and bands were quantified using the UVItec Alliance detection system.

In vivo delivery of VEsiCas. Animal care and treatments were conducted in conformity with institutional guidelines in compliance with national and international laws and policies (EEC Council Directive 86/609, OJL 358, Dec. 12, 1987 and D.lgs 116/92), upon approval by the ICGEB Ethical Committee for Animal Experimentation and by the Italian Ministry of Health. Transgenic mice (males) expressing EGFP (C57BL/6-Tg(CAG-EGFP)1Osb/J from Jackson Laboratories) at post-natal day 5 were anesthetized by hypothermia on ice for ˜3-5 min, placing a gauze below the pup to avoid direct contact with ice. A transverse skin incision across the lower half of the chest was performed using small scissors, followed by gentle separation of the skin from underlying muscle by using blunt dissection. A lateral thoracotomy was created by making a small incision at the fourth intercostal space to visualize the heart. VEsiCas (5 microliters, corresponding to 4 μg of SpCas9) carrying a control sgRNA (sgCtr) or a guide targeting EGFP (sgEGFPBi) were injected into the left ventricular anterior wall using a 31G needle. The ribs and the skin were sutured together using a 8-0 nonabsorbable Prolene suture to seal the chest wall incision. The neonates were warmed rapidly under a heat lamp for several minutes until recovery and re-introduced to the mother. After 10 days, the hearts were collected, fixed in 4% paraformaldehyde and snap frozen in isopentane/liquid nitrogen for fluorescence microscopy analysis.

Immunofluorescence. Frozen sections were washed 3 times in PBS, permeabilised in 0.5% Triton X-100 for 30 minutes and blocked in 10% goat serum for 1 hour. Sections were stained overnights at 4° C. with anti-sarcomeric α-actinin antibodies (Abcam), 1:100 in 5% goat serum. After 2 washing steps of 5 minutes in 0.5% Triton X-100 at room temperature, sections were incubated 1 hour in 1:200 anti-mouse secondary antibody conjugated to Alexa Fluor-594 (Life Technologies) in 10% goat serum for 45 minutes at room temperature. Nuclei were stained with 4′,6-Diamidino-2-phenylindole dihydrochloride (DAPI) solution (Sigma).

### Comparative Example

Side-by-Side Evaluation of VEsiCas and Other State-of-the-Art Systems

To demonstrate the superior editing performance of VEsiCas, we conducted a side-by-side comparison with state-of-the-art technologies, namely vesicles produced using a U6-based nuclear transcription system for the sgRNA (U6-vesicles). U6-vesicles can be considered a good proxy for all the technologies already present in the art, as they all rely on U6 driven nuclear transcription of the sgRNA. We produced batches of VEsiCas and U6-vesicles incorporating a sgRNA directed towards the EGFP coding sequence (sgRNA EGFPBi) and measured EGFP knockout in a reporter cell line stably expressing EGFP (HEK293-EGFP). The raw results are reported in FIG. 12a, while a graph in which data are normalized on the amount of SpCas9 used for the transduction is shown in FIG. 12b. Quantification of SpCas9 content in each sample was obtained by western blot, as described in the Methods. VEsiCas demonstrated higher levels of EGFP knock out, with an overall increase in the total number of edited cells of 1,6-fold.

To further broaden our comparison, we also evaluated the editing efficacy of VEsiCas in parallel with Gesicles (disclosed in WO2015/191911A2), a commercial vesicle system distributed by Takara-Clontech able to deliver SpCas9-sgRNA complexes, and U6-vesicles. We produced VEsiCas together with Gesicles and U6-vesicles, incorporating the EGFPBi sgRNA in all three preparations. We treated HEK293-EGFP cells using fractions of the different vesicle preparations containing the same amount of incorporated SpCas9 (40 ng) and measured the decrease in fluorescence. As shown in FIG. 13, VEsiCas is outperforming both U6-vesicles (1.6-fold more editing), as expected, and Gesicles (1.5-fold increased knockout), demonstrating the highest levels of EGFP knockout. Interestingly, the editing levels obtained with U6-vesicles and Gesicles are similar, further supporting the notion that the way in which the sgRNA is transcribed in producer cells is fundamental to determine the editing efficacy of the vesicle preparation, more than other technical characteristics of the vesicles itself. Overall, since the amount of SpCas9 protein used is the same, the increased activity of VEsiCas compared to standard methods must be caused by an increased amount of sgRNA incorporated in the vesicles.

Evaluation of sgRNA Incorporation by VEsiCas and State-of-the-Art Systems

In order to better characterize the efficacy of our T7-driven cytoplasmic transcription system, we compared the levels of sgRNA incorporated in U6-vesicles (nuclear PolIII-expressed sgRNA) with those incorporated in VEsiCas (cytoplasmic T7 RNA polymerase-derived sgRNA). The guide RNA used is designed to target the EGFP coding sequence (EGFPBi sgRNA). We evaluated the sgRNA content in each type of vesicle preparation using an RT-qPCR based assay, as reported in the Methods section. Total RNA was extracted from the vesicle preparation using the Nucleospin miRNA kit (Macherey-Nagel) according to the manufacturer's protocol. A standard curve for absolute quantification was prepared using an in vitro transcribed sgRNA molecule identical to the one incorporated into both vesicle preparations (FIG. 14a). As shown in FIG. 14b, a higher amount of gRNA was measured in VEsicas compared to U6-vescicles (1.6-fold more). This value can be further corrected by taking into account the total amount of SpCas9 incorporated in the vesicles (measured as indicated in the Methods section) to correct for production biases. These calculations confirm higher incorporation for T7-transcribed sgRNAs, increasing the ratio over U6-driven expression to 1.7-fold (FIG. 14c).

Evaluation of the Molar Ratio Between SpCas9 and sgRNA in VEsiCas

Since the amount of incorporated sgRNA correlates with higher editing efficiency, we reasoned that an unexpected advantage of VEsiCas is represented by the increased level of SpCas9 complexed with its sgRNA and incorporated into the vesicles. To measure the molar ratio between incorporated SpCas9 and incorporated sgRNA, we quantified the two molecules present in a VEsiCas preparation. For SpCas9 quantification we used western blot (FIG. 15a), as reported in the Methods section. To increase the reliability of the measurement we loaded two different standard curves obtained with two different preparations of recombinant SpCas9 (shown in FIG. 15b-c). As shown in Table 3, the quantifications using the two curves are consistent.

TABLE 3
Absolute quantification of SpCas9 incorporated
into VEsiCas using replicated standard curves

| | Cas9 | Cas9 |
|---|---|---|
| | (ng) | (pmol) |
| VEsiCas (Standard 1) | 16.59 | 0.104 |
| VEsiCas (Standard 2) | 22.73 | 0.142 |

We next extracted total RNA from the VEsiCas preparation and quantified the amount of sgRNA by using the RT-qPCR assay described in the Methods and in the previous paragraph. From these absolute quantifications we obtained the molar ratio between the two molecules, which can be intended as the amount of sgRNA incorporated by the nuclease. As reported Table 4 the ratio is equal to 1:0.85, indicating high complexing efficiency thanks to the T7 RNA polymerase-based cytoplasmic transcription system.

TABLE 4
Molar ratio between SpCas9 and sgRNA in
VEsiCas

| | Cas9 | Cas9 | sgRNA | sgRNA | Cas9:sgRNA |
|---|---|---|---|---|---|
| | (ng) | (pmol) | (ng) | (pmol) | molar ratio |
| VEsiCas | 19.66 | 0.123 | 3.58 | 0.105 | 1:0.85 |

### REFERENCES

Accola, M. A., Strack, B. & Gottlinger, H. G., 2000. Efficient particle production by minimal Gag constructs which retain the carboxy-terminal domain of human immunodeficiency virus type 1 capsid-p2 and a late assembly domain. Journal of Virology, 74(12), pp. 5395-5402.
Aubertin, K. et al., 2016. Massive release of extracellular vesicles from cancer cells after photodynamic treatment or chemotherapy. Scientific Reports, 6(1), p. 35376.
Bewicke-Copley, F. et al., 2017. Extracellular vesicles released following heat stress induce bystander effect in unstressed populations. Journal of extracellular vesicles, 6(1), p. 1340746.
Brinkman, E. K. et al., 2014. Easy quantitative assessment of genome editing by sequence trace decomposition. Nucleic Acids Research, 42(22), pp. e168-e168.
Buchholz, U. J., Finke, S. & Conzelmann, K. K., 1999. Generation of bovine respiratory syncytial virus (BRSV) from cDNA: BRSV NS2 is not essential for virus replication in tissue culture, and the human RSV leader region acts as a functional BRSV genome promoter. Journal of Virology, 73(1), pp. 251-259.
Casini, A. et al., 2015. Reduction of HIV-1 infectivity through endoplasmic reticulum-associated degradation-mediated Env depletion. Journal of Virology, 89(5), pp. 2966-2971.
Chew, W. L. et al., 2016. A multifunctional AAV—CRISPR-Cas9 and its host response. Nature methods, 13(10), pp. 868-874.
Chick, H. E. et al., 2012. Integrase-deficient lentiviral vectors mediate efficient gene transfer to human vascular smooth muscle cells with minimal genotoxic risk. Human Gene Therapy, 23(12), pp. 1247-1257.
Choi, J. G. et al., 2016. Lentivirus pre-packed with Cas9 protein for safer gene editing. Gene Therapy, 23(7), pp. 627-633.
Chylinski, K., Le Rhun, A. & Charpentier, E., 2013. The tracrRNA and Cas9 families of type II CRISPR-Cas immunity systems. RNA Biology, 10(5), pp. 726-737.
Cox, D. B. T. et al., 2017. RNA editing with CRISPR-Cas13. Science, 358(6366), pp. 1019-1027.
Dahlman, J. E. et al., 2015. Orthogonal gene knockout and activation with a catalytically active Cas9 nuclease. Nature Biotechnology, 33(11), pp. 1159-1161.
Dang, Y. et al., 2015. Optimizing sgRNA structure to improve CRISPR-Cas9 knockout efficiency. Genome Biology, pp. 1-10.
Deyle, D. R. & Russell, D. W., 2009. Adeno-associated virus vector integration. Current opinion in molecular therapeutics, 11(4), pp. 442-447.
Diao, Y. et al., 2016. A new class of temporarily phenotypic enhancers identified by CRISPR/Cas9-mediated genetic screening. Genome Research, 26(3), pp. 397-405.
Eaton, H. E. et al., 2017. African Swine Fever Virus NP868R Capping Enzyme Promotes Reovirus Rescue during Reverse Genetics by Promoting Reovirus Protein Expression, Virion Assembly, and RNA Incorporation into Infectious Virions. S. López, ed. Journal of Virology, 91(11), pp. e02416-16.
Elroy-Stein, O. & Moss, B., 2001. Gene expression using the vaccinia virus/T7 RNA polymerase hybrid system. Current protocols in protein science, Chapter 5, pp. Unit5.15-5.15.11.
Esvelt, K. M., Carlson, J. C. & Liu, D. R., 2011. A system for the continuous directed evolution of biomolecules. Nature, 472(7344), pp. 499-503.
Friedland, A. E. et al., 2015. Characterization of Staphylococcus aureus Cas9: a smaller Cas9 for all-in-one adeno-associated virus delivery and paired nickase applications. Genome Biology, 16(1), p. 257.
Fu, Y. et al., 2013. High-frequency off-target mutagenesis induced by CRISPR-Cas nucleases in human cells. Nature Biotechnology, pp. 1-6.
Fuchs, A.-L., Neu, A. & Sprangers, R., 2016. A general method for rapid and cost-efficient large-scale production of 5′ capped RNA. RNA, 22(9), pp. 1454-1466.
Gabriel, R. et al., 2011. An unbiased genome-wide analysis of zinc-finger nuclease specificity. Nature Biotechnology, 29(9), pp. 816-823.
Gagnon, J. A. et al., 2014. Efficient mutagenesis by Cas9 protein-mediated oligonucleotide insertion and large-scale assessment of single-guide RNAs. B. Riley, ed. PLoS ONE, 9(5), p. e98186.
Gao, L. et al., 2017. Engineered Cpf1 variants with altered PAM specificities. Nature Biotechnology, 35(8), pp. 789-792.
Gao, Y. et al., 2016. Complex transcriptional modulation with orthogonal and inducible dCas9 regulators. Nature methods, 13(12), pp. 1043-1049.
Geer, L. Y. et al., 2002. CDART: protein homology by domain architecture. Genome Research, 12(10), pp. 1619-1623.
Habjan, M. et al., 2008. T7 RNA polymerase-dependent and -independent systems for cDNA-based rescue of Rift Valley fever virus. The Journal of general virology, 89(Pt 9), pp. 2157-2166
Hou, Z. et al., 2013. Efficient genome engineering in human pluripotent stem cells using Cas9 from Neisseria meningitidis. Proceedings of the National Academy of Sciences of the United States of America, 110(39), pp. 15644-15649.
Hu, J. H. et al., 2018. Evolved Cas9 variants with broad PAM compatibility and high DNA specificity. Nature, 556(7699), pp. 57-63.
Jordan, A., Bisgrove, D. & Verdin, E., 2003. HIV reproducibly establishes a latent infection after acute infection of T cells in vitro. The EMBO Journal, 22(8), pp. 1868-1877. Available at: http://www.pubmedcentral.nih.gov/articlerender.fcgi?artid=154479&tool=pmcentrez&rendertype=abstract.
Kim, D.-H. et al., 2004. Interferon induction by siRNAs and ssRNAs synthesized by phage polymerase. Nature Biotechnology, 22(3), pp. 321-325.
Kim, S. et al., 2014. Highly efficient RNA-guided genome editing in human cells via delivery of purified Cas9 ribonucleoproteins. Genome Research, 24(6), pp. 1012-1019.
Kleinstiver, B. P. et al., 2016. High-fidelity CRISPR-Cas9 nucleases with no detectable genome-wide off-target effects. Nature, 529(7587), pp. 490-495.
Komor, A. C., Badran, A. H. & Liu, D. R., 2017. CRISPR-Based Technologies for the Manipulation of Eukaryotic Genomes. Cell, 168(1-2), pp. 20-36.
Lapinaite, A., Doudna, J. A. & Cate, J. H. D., 2018. Programmable RNA recognition using a CRISPR-associated Argonaute. Proceedings of the National Academy of Sciences of the United States of America, 115(13), pp. 3368-3373.
Liu, K. I. et al., 2016. A chemical-inducible CRISPR-Cas9 system for rapid control of genome editing. Nature chemical biology, 12(11), pp. 980-987.
Lombardo, A. et al., 2007. Gene editing in human stem cells using zinc finger nucleases and integrase-defective lentiviral vector delivery. Nature Biotechnology, 25(11), pp. 1298-1306.
Long, C. et al., 2016. Postnatal genome editing partially restores dystrophin expression in a mouse model of muscular dystrophy. Science, 351(6271), pp. 400-403.
Mangeot, P.-E. et al., 2011. Protein transfer into human cells by VSV-G-induced nanovesicles. Molecular therapy: the journal of the American Society of Gene Therapy, 19(9), pp. 1656-1666.
Mout, R., Ray, M., Lee, Y.-W., et al., 2017. In Vivo Delivery of CRISPR/Cas9 for Therapeutic Gene Editing: Progress and Challenges. Bioconjugate chemistry, 28(4), pp. 880-884.
Mout, R., Ray, M., Yesilbag Tonga, G., et al., 2017. Direct Cytosolic Delivery of CRISPR/Cas9-Ribonucleoprotein for Efficient Gene Editing. ACS nano, 11(3), pp. 2452-2458.
Nakai, H. et al., 2001. Extrachromosomal recombinant adeno-associated virus vector genomes are primarily responsible for stable liver transduction in vivo. Journal of Virology, 75(15), pp. 6969-6976.
Nihongaki, Y. et al., 2015. Photoactivatable CRISPR-Cas9 for optogenetic genome editing. Nature Biotechnology, 33(7), pp. 755-760.
Nissim, L. et al., 2014. Multiplexed and programmable regulation of gene networks with an integrated RNA and CRISPR/Cas toolkit in human cells. Molecular Cell, 54(4), pp. 698-710.
Nuñez, J. K., Harrington, L. B. & Doudna, J. A., 2016. Chemical and Biophysical Modulation of Cas9 for Tunable Genome Engineering. ACS Chemical Biology, 11(3), pp. 681-688.
Perez-Pinera, P. et al., 2013. RNA-guided gene activation by CRISPR-Cas9-based transcription factors. Nature methods, 10(10), pp. 973-976.
Petris, G. et al., 2017. Hit and go CAS9 delivered through a lentiviral based self-limiting circuit. Nature Communications, 8, p. 15334.
Pichlmair, A. et al., 2006. RIG-I-mediated antiviral responses to single-stranded RNA bearing 5′-phosphates. Science, 314(5801), pp. 997-1001.
Ramakrishna, S. et al., 2014. Gene disruption by cell-penetrating peptide-mediated delivery of Cas9 protein and guide RNA. Genome Research, 24(6), pp. 1020-1027.
Ramqvist, T., Andreasson, K. & Dalianis, T., 2007. Vaccination, immune and gene therapy based on virus-like particles against viral infections and cancer. Expert opinion on biological therapy, 7(7), pp. 997-1007.
Ran, F. A. et al., 2013. Double Nicking by RNA-Guided CRISPR Cas9 for Enhanced Genome Editing Specificity. Cell, 154(6), pp. 1380-1389.
Ran, F. A. et al., 2015. In vivo genome editing using Staphylococcus aureus Cas9. Nature, 520(7546), pp. 186-191.
Rittner, K. et al., 2002. New basic membrane-destabilizing peptides for plasmid-based gene delivery in vitro and in vivo. Molecular Therapy, 5(2), pp. 104-114.
Ruozi, G. et al., 2015. AAV-mediated in vivo functional selection of tissue-protective factors against ischaemia. Nature Communications, 6(1), p. 7388.
Samuel, P. et al., 2018. Cisplatin induces the release of extracellular vesicles from ovarian cancer cells that can induce invasiveness and drug resistance in bystander cells. Philosophical transactions of the Royal Society of London. Series B, Biological sciences, 373(1737), p. 20170065.
Schoderboeck, L. et al., 2015. Chimeric rabies SADB19-VSVg-pseudotyped lentiviral vectors mediate long-range retrograde transduction from the mouse spinal cord. Gene Therapy, 22(5), pp. 357-364.
Shmakov, S. et al., 2015. Discovery and Functional Characterization of Diverse Class 2 CRISPR-Cas Systems. Molecular Cell, 60(3), pp. 385-397.
Slaymaker, I. M. et al., 2016. Rationally engineered Cas9 nucleases with improved specificity. Science, 351(6268), pp. 84-88.
Suzuki, K. et al., 2016. In vivo genome editing via CRISPR/Cas9 mediated homology-independent targeted integration. Nature, 540(7631), pp. 144-149.
Tak, Y. E. et al., 2017. Inducible and multiplex gene regulation using CRISPR-Cpf1-based transcription factors. Nature methods, 14(12), pp. 1163-1166.
Thyme, S. B. et al., 2016. Internal guide RNA interactions interfere with Cas9-mediated cleavage. Nature Communications, 7, p. 11750.
Truong, D.-J. J. et al., 2015. Development of an intein-mediated split-Cas9 system for gene therapy. Nucleic Acids Research, 43(13), pp. 6450-6458.
Tsai, S. Q. & Joung, J. K., 2016. Defining and improving the genome-wide specificities of CRISPR-Cas9 nucleases. Nature Reviews Genetics, 17(5), pp. 300-312.
Tsai, S. Q. et al., 2014. GUIDE-seq enables genome-wide profiling of off-target cleavage by CRISPR-Cas nucleases. Nature Biotechnology, 33(2), pp. 187-197.
Tsai, S. Q. et al., 2016. Open-source guideseq software for analysis of GUIDE-seq data. Nature Biotechnology, 34(5), pp. 483-483.
Voelkel, C. et al., 2010. Protein transduction from retroviral Gag precursors. Proceedings of the National Academy of Sciences of the United States of America, 107(17), pp. 7805-7810.
Vora, S. et al., 2016. Next stop for the CRISPR revolution: RNA-guided epigenetic regulators. The FEBS Journal, 283(17), pp. 3181-3193.
Wang, X. et al., 2015. Unbiased detection of off-target cleavage by CRISPR-Cas9 and TALENs using integrase-defective lentiviral vectors. Nature Biotechnology, 33(2), pp. 175-178.
Wei, C. M. & Moss, B., 1975. Methylated nucleotides block 5′-terminus of vaccinia virus messenger RNA. Proceedings of the National Academy of Sciences, 72(1), pp. 318-322.
Wright, A. V. et al., 2015. Rational design of a split-Cas9 enzyme complex. Proceedings of the National Academy of Sciences, 112(10), pp. 2984-2989.
Yan, W. X. et al., 2018. Cas13d Is a Compact RNA-Targeting Type VI CRISPR Effector Positively Modulated by a WYL-Domain-Containing Accessory Protein. Molecular Cell, 70(2), pp. 327-339.e5.
Yang, Y. et al., 2016. A dual AAV system enables the Cas9-mediated correction of a metabolic liver disease in newborn mice. Nature Biotechnology, 34(3), pp. 334-338.
Zacchigna, S., Zentilin, L. & Giacca, M., 2014. Adeno-associated virus vectors as therapeutic and investigational tools in the cardiovascular system. Circulation research, 114(11), pp. 1827-1846.
Zetsche, B., Volz, S. E. & Zhang, F., 2015. A split-Cas9 architecture for inducible genome editing and transcription modulation. Nature Biotechnology, 33(2), pp. 139-142.
Zhang, J.-P. et al., 2016. Different Effects of sgRNA Length on CRISPR-mediated Gene Knockout Efficiency. Scientific Reports, 6(1), p. 28566.
Zuris, J. A. et al., 2015. Cationic lipid-mediated delivery of proteins enables efficient protein-based genome editing in vitro and in vivo. Nature Biotechnology, 33(1), pp. 73-80.

1. A vesicle comprising:
i) a lipid envelope associated with at least one membrane-associated protein;
ii) at least one guide RNA molecule; and
iii) optionally at least one RNA-guided nuclease;
wherein the vesicle has at least one of the following features:
the at least one guide RNA molecule is present within the vesicle in an amount of at least 0.2% w/w to total vesicle protein mass; and
if the vesicle contains also the at least one RNA-guided nuclease, the at least one guide RNA molecule is complexed to the at least one RNA-guided nuclease, wherein the at least one guide RNA molecule is present within the vesicle in a molar ratio with respect to the at least one RNA-guided nuclease equal or above to 0.85:1.

2. The vesicle according to claim 1, wherein the lipid envelope is selected from a mono- or bi-layer lipid structure, an exosome, an enveloped virus, an enveloped viral-like particle, a microsome, an endosome, a nanosome, a vacuole, preferably an exosome or an enveloped viral-like particle.

3. The vesicle according to claim 1 or claim 2, wherein the at least one membrane-associated protein stimulates vesicle formation from a vesicle producing cell and/or mediates vesicle fusion to a target cell.

4. The vesicle according any one of the previous claims, wherein the at least one membrane-associated protein is selected from: Clatrin adaptor complex AP1, proteolipid protein PLP1, TSAP6, CHMP4C, VSV-G envelope protein, ALV envelope, BRL envelope glycoprotein, rabies virus envelope glycoprotein, influenza NA/HA/M2 envelope protein, MuLV amphotropic envelope, baculovirus gp64, HIV gp160; capsid proteins, nucleocapsid proteins, matrix protein of enveloped viruses having an interaction with the cell membrane; ebola VP40, ebola glycoprotein, Gag and/or Gag-pol retroviral protein, Gag and/or Gag-Pol lentiviral protein, Arc proteins, TY3/gypsy retrotransposons envelope proteins, HIV-1 Vpu, minimal-Gag, SADB19-VSV-G fusion, a portion of the VSV-G transmembrane domain and/or its intracellular domain and/or its extracellular domain fused with one of the envelope proteins listed above, single chain variable antibody fragments (scFv) derived from immunoglobulin variable domains able to recognize surface molecules on target cells, protein receptors able to recognize surface molecules on target cells, proteinaceous ligands able to recognize surface molecules on target cells and analogues thereof.

5. The vesicle according to any one of the previous claims, wherein the at least one guide RNA molecule is selected from sgRNA, crRNA, tracrRNA, miRNA, shRNA, siRNA.

6. The vesicle according to any one of the previous claims, wherein the at least one RNA-guided nuclease is selected from CRISPR class 2 type-II, type-V and/or type-VI nucleases or Argonaute RNA-guided nucleases and variants thereof.

7. The vesicle according to claim 6, wherein the at least one RNA-guided nuclease is selected from: a Cas9, Cpf1, Cas13 and Ago2 nuclease and variants thereof; a Cas9, Cpf1 and Cas13 nuclease mutant with or without nuclease activity; a Cas9 and Cpf1 nuclease mutant with nickase activity; and a Cas9 and Cpf1 nuclease fused to a protein domain selected from: protein tags, additional nuclease domains, nucleic acid-editing domains, cell penetrating peptides, peptides allowing endosomal escape, transcriptional regulators, chromatin regulators, proteins or protein domains modulating DNA repair, proteins or protein domains allowing post-translational modification of other proteins, protein domains or peptides regulating protein stability and/or localization inside the cell, protein-binding domains.

8. The vesicle according to claim 7, wherein the at least one guide RNA molecule, forming a complex with a Cas9, Cpf1 or Cas13 nuclease and a variant thereof, or a Cas9, Cpf1 or Cas13 nuclease mutant with or without nuclease activity, is engineered to include an aptamer, preferably a MS2 aptamer, having interacting properties with an aptamer interacting protein domain, preferably a MS2 protein, which is fused to other protein domains encoding a base editor, a transcriptional regulator or a chromatin regulator.

9. The vesicle according to any one of the previous claims, wherein the at least one RNA-guided nuclease is fused to at least one of: a farnesylation signal, a myristoylation signal, a transmembrane domain.

10. The vesicle according to any one of the previous claims, wherein the vesicle, once delivered to a target cell, provides for transient expression of the RNA-guided nuclease and/or guide RNA within the target cell in order to minimize cell toxicity.

11. A method for producing the vesicle according to any one of claims 1 to 10, wherein the method comprises the following steps:
i) providing a packaging cell, wherein the packaging cell has the following features:
a) the cell is tolerant to high levels of direct cytosolic transcription, wherein the cell tolerance is determined by: a lack of expression in the cell of at least one RNA virus-sensing pathway selected from: RIG-I, RIG-I-like protein, MDA-5, IPS-1, RIPI, FADD, TRAF6, TRAF3, TANK, NAP, NEMO, IKKα, IKKβ, IKKε, IKKγ, TBK1, DDX3, IκB, NF-κB, IRF3, IRF7, p65, p50, RIP1, TLR3, TLR7, TLR8, INF-α, INF-γ1, INF-γ2, INF-γ3, Caspase-1, TLR8; and/or a cell ability to cap at least one guide-RNA molecule by expression of capping enzymes; and/or a cell ability to express a 5′-phosphatase for de-phosphorylating 5′-triphosphate transcripts; and
b) the cell stably or transiently expresses an RNA polymerase in the cytoplasm;
ii) transfecting the packaging cell with at least one first expression cassette, and at least one second expression cassette and optionally at least one third expression cassette, wherein:
a) the first expression cassette comprises a first nucleotide sequence to transcribe at least one guide RNA molecule;
b) the second expression cassette comprises a second nucleotide sequence encoding at least one membrane-associated protein; and
c) the third expression cassette comprises a third nucleotide sequence encoding at least one RNA-guided nuclease;
iii) producing the vesicle from the packaging cells.

12. The method according to claim 11, wherein the RNA polymerase is a bacteriophage RNA polymerase selected from: RNA polymerase of phage T7, RNA polymerase of Bacteriophage SP6, RNA polymerase of Yersinia pestis bacteriophage phiA1122, RNA polymerase of Pseudomonas bacteriophage gh-1, RNA polymerase of Pseudomonas putida bacteriophage, RNA polymerase of Bacteriophage T3, RNA polymerase of Bacteriophage T4, RNA polymerase of Roseophage SI01, RNA polymerase of Bacteriophage phiYe03-12, RNA polymerase of bacteriophage phiKMV, RNA polymerase of Enterobacteria bacteriophage K1-5, RNA polymerase of Vibriophage VpV262, RNA polymerase of BA14, RNA polymerase of BA127 and RNA polymerase of BA156, and variants thereof.

13. The method according to claim 11 or claim 12, wherein the packaging cell is selected from BHK21, BSR-T7/5, BHK-T7 and Vero cells.

14. The method according to anyone of claims 11 to 13, wherein the at least one membrane-associated protein is selected from: Clatrin adaptor complex AP1, proteolipid protein PLP1, TSAP6, CHMP4C, VSV-G envelope protein, ALV envelope, BRL envelope glycoprotein, rabies virus envelope glycoprotein, influenza NA/HA/M2 envelope protein, MuLV amphotropic envelope protein, baculovirus gp64, HIV gp160, capsid proteins, nucleocapsid proteins, matrix protein of enveloped viruses having an interaction with the cell membrane, ebola VP40, ebola glycoprotein, Gag and/or Gag-pol retroviral protein, Gag and/or Gag-Pol lentiviral protein, Arc proteins, TY3/gypsy retrotransposons envelope proteins, HIV-1 Vpu, minimal-Gag, SADB19-VSV-G fusion, a portion of the VSV-G transmembrane domain and/or its intracellular domain and/or its extracellular domain fused with one of the envelope proteins listed above, single chain variable antibody fragments (scFv) derived from immunoglobulin variable domains able to recognize surface molecules on target cells, protein receptors able to recognize surface molecules on target cells, proteinaceous ligands able to recognize surface molecules on target cells and analogues thereof.

15. The method according to anyone of claims 11 to 14, wherein the at least one RNA-guided nuclease is selected from: CRISPR class 2 type-II, type-V and type-VI nucleases and Argonaute RNA-guided nucleases.

16. The method according to anyone of claims 11 to 15, wherein the at least one RNA-guided nuclease is selected from: a Cas9, Cpf1, Cas13 and Ago2 nuclease and variants thereof; a Cas9, Cpf1 and Cas13 nuclease mutant with or without nuclease activity; a Cas9 and Cpf1 nuclease mutant with nickase activity; a Cas9 and Cpf1 nuclease fused to a protein domain selected from: amino acid sequences that encode protein tags, additional nuclease domains, nucleic acid-editing domains, cell penetrating peptides and peptides allowing endosomal escape, transcriptional regulators, chromatin regulators, proteins or protein domains modulating DNA repair, proteins or protein domains allowing post-translational modification of other proteins, protein domains or peptides regulating protein stability and/or localization inside the cell, protein-binding domains.

17. The method according to claim 16, wherein the at least one guide RNA molecule, forming a complex with a Cas9, Cpf1 or Cas13 nuclease, or a Cas9, Cpf1 or Cas13 nuclease mutant with or without nuclease activity, is engineered to include an aptamer, preferably a MS2 aptamer, having interacting properties with a protein domain selected from: a base editor, a transcriptional regulator or a chromatin regulator.

18. The method according to anyone of claims 11 to 17, wherein the step ii) provides for transfecting the packaging cell with at least one further expression cassette, wherein the least one further expression cassette comprises a further nucleotide sequence encoding at least one membrane-associated protein, useful to change vesicles tropism to target cells, selected from: HIV gp160, HIV gp120, VSV-G, ALV envelope, ebola glycoprotein, BRL envelope glycoprotein, rabies virus envelope glycoprotein, influenza NA/HA/M2, MuLV amphotropic envelope, baculovirus gp64, TCR-alpha, CD4, MHC-I, MHC-II, variable domains of antibodies and/or full-length antibodies that recognize surface molecules on target cells,
with proviso that the at least one membrane-associated protein encoded by the further nucleotide sequence comprised in the further expression cassette is different from the at least one membrane-associated protein encoded by the second nucleotide sequence comprised in the second expression cassette.

19. The method according to anyone of claims 11 to 17, wherein the third nucleotide sequence encoding at least one RNA-guided nuclease further contains at least one nucleotide sequence encoding at least one of: a farnesylation signal, a myristoylation signal, a transmembrane domain.


SEQUENCE LISTING (55 sequences)

SEQ ID NO: 1 - DNA, 25 bp, artificial
  EGFP5 oligo 1
tataggaagttcgagggcgacaccc

SEQ ID NO: 2 - DNA, 27 bp, artificial
  EGFP5 oligo 2
aaacgggtgtcgccctcgaacttcccc

SEQ ID NO: 3 - DNA, 24 bp, artificial
  EGFPBi oligo 1
tatagggcacgggcagcttgccgg

SEQ ID NO: 4 - DNA, 26 bp, artificial
  EGFPBi oligo 2
aaacccggcaagctgcccgtgccccc

SEQ ID NO: 5 - DNA, 27 bp, artificial
  EGFP3gW oligo 1
tatagggctcgtgaccaccctgaccta

SEQ ID NO: 6 - DNA, 29 bp, artificial
  EGFP3gW oligo 2
aaactaggtcagggtggtcacgagccccc

SEQ ID NO: 7 - DNA, 24 bp, artificial
  GFPI2 oligo 1
tataggtgggcaccggcttccccg

SEQ ID NO: 8 - DNA, 26 bp, artificial
  GFPI2 oligo 2
aaaccggggaagccggtgcccacccc

SEQ ID NO: 9 - DNA, 24 bp, artificial
  VEGFA site3 oligo 1
tataggtgagtgagtgtgtgcgtg

SEQ ID NO: 10 - DNA, 24 bp, artificial
  VEGFA site3 oligo 2
aaaccacgcacacactcactcacc

SEQ ID NO: 11 - DNA, 25 bp, artificial
  CXCR4 oligo 1
tataggaagcgtgatgacaaagagg

SEQ ID NO: 12 - DNA, 27 bp, artificial
  CXCR4 oligo 2
aaaccctctttgtcatcacgcttcccc

SEQ ID NO: 13 - DNA, 29 bp, artificial
  EGFP5 target site
ggtgaagttcgagggcgacaccctggtga

SEQ ID NO: 14 - DNA, 29 bp, artificial
  EGFPBi target site
ccaccggcaagctgcccgtgccctggccc

SEQ ID NO: 15 - DNA, 29 bp, artificial
  EGFP3gW target site
accctcgtgaccaccctgacctacggcgt

SEQ ID NO: 16 - DNA, 29 bp, artificial
  GFPI2 target site
ggtggtgggcaccggcttccccgaggaca

SEQ ID NO: 17 - DNA, 29 bp, homo sapiens
gtgggtgagtgagtgtgtgcgtgtggggt

SEQ ID NO: 18 - DNA, 29 bp, homo sapiens
gtgagtgagtgagtgtgtgtgtggggggg

SEQ ID NO: 19 - DNA, 29 bp, homo sapiens
atgtgtgggtgagtgtgtgcgtgaggaca

SEQ ID NO: 20 - DNA, 27 bp, homo sapiens
aagggaagcgtgatgacaaagaggagg

SEQ ID NO: 21 - DNA, 35 bp, artificial
  T7 fw - HindIII
actaagcttgtcgaccatgaacacgattaacatcg

SEQ ID NO: 22 - DNA, 27 bp, artificial
  T7 rev - XbaI
taatctagattacgcgaacgcgaagtc

SEQ ID NO: 23 - DNA, 24 bp, enterobacteria phage T7
gaaattaatacgactcactatagg

SEQ ID NO: 24 - DNA, 19 bp, artificial
  gRNA end rev
aagcaccgactcggtgcca

SEQ ID NO: 25 - DNA, 23 bp, artificial
  EGFP fw
accatggtgagcaagggcgagga

SEQ ID NO: 26 - DNA, 24 bp, artificial
  EGFP rev
agctcgtccatgccgagagtgatc

SEQ ID NO: 27 - DNA, 22 bp, homo sapiens
gcatacgtgggctccaacaggt

SEQ ID NO: 28 - DNA, 22 bp, homo sapiens
ccgcaatgaaggggaagctcga

SEQ ID NO: 29 - DNA, 21 bp, homo sapiens
caggcgccttgggctccgtca

SEQ ID NO: 30 - DNA, 20 bp, homo sapiens
ccccaggatccgcgggtcac

SEQ ID NO: 31 - DNA, 23 bp, homo sapiens
agtcagccctctgtatccctgga

SEQ ID NO: 32 - DNA, 25 bp, homo sapiens
gagatatctgcaccctcatgttcac

SEQ ID NO: 33 - DNA, 32 bp, homo sapiens
agaggagttagccaagatgtgactttgaaacc

SEQ ID NO: 34 - DNA, 33 bp, homo sapiens
ggacaggatgacaataccaggcaggataaggcc

SEQ ID NO: 35 - DNA, 26 bp, artificial
  TetO oligo 1
tataggtacgttctctatcactgata

SEQ ID NO: 36 - DNA, 26 bp, artificial
  TetO oligo 2
aaactatcagtgatagagaacgtacc

SEQ ID NO: 37 - DNA, 29 bp, artificial
  TetO target site
acatacgttctctatcactgatagggagt

SEQ ID NO: 38 - DNA, 24 bp, artificial
  Ctr oligo 1
tataggagacggattgacgtctct

SEQ ID NO: 39 - DNA, 24 bp, artificial
  Ctr oligo 2
aaacagagacgtcaatccgtctcc

SEQ ID NO: 40 - DNA, 2652 bp, enterobacteria phage T7
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 41 - protein, 883 aa, enterobacteria phage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 42 - DNA, 2625 bp, enterobacteria phage SP6
atgcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagagcgctggatggttgatacaggtatcgaagtacctgagcaaggggagttcgaccttaacgaaatcatggattctgaatacgtatttgcctaa

SEQ ID NO: 43 - protein, 874 aa, enterobacteria phage SP6
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 44 - DNA, 78 bp, artificial
  sgRNA scaffold
gttttagagctagaaatagcaagttaaaataaggctagtccgttatcaacttgaaaaagtggcaccgagtcggtgctt

SEQ ID NO: 45 - DNA, 88 bp, artificial
  optimised sgRNA scaffold 1
gtttaagagctatgctggaaacagcatagcaagtttaaataaggctagtccgttatcaacttgaaaaagtggcaccgagtcggtgctt

SEQ ID NO: 46 - DNA, 88 bp, artificial
  optimised sgRNA scaffold 2
gtttcagagctatgctggaaacagcatagcaagtttaaataaggctagtccgttatcaacttgaaaaagtggcaccgagtcggtgctt

SEQ ID NO: 47 - DNA, 5890 bp, artificial
  Gag-SpCas9 cds
atgggtgcgagagcgtcggtattaagcgggggagaattagatcgatgggaaaaaattcggttaaggccagggggaaagaagaagtacaagctaaagcacatcgtatgggcaagcagggagctagaacgattcgcagttaatcctggcctgttagaaacatcagaaggctgtagacaaatactgggacagctacaaccatcccttcagacaggatcagaggagcttcgatcactatacaacacagtagcaaccctctattgtgtgcaccagcggatcgagatcaaggacaccaaggaagctttagacaagatagaggaagagcaaaacaagtccaagaagaaggcccagcaggcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaggacctgaacacgatgttgaacaccgtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagagatctacaggaggtggataatcctgggattgaacaagatcgtgaggatgtatagccctaccagcattctggacataagacaaggaccaaaagaaccctttagagactatgtagaccggttctataaaactctaagagctgagcaagcttcacaggaggtaaaaaattggatgacagaaaccttgttggtccaaaatgcgaacccagattgtaagaccatcctgaaggctctcggcccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacgagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcagcgacccctcgtcacaaggcgcgcaaggaagccagaactatccaatcgtccagaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctc

SEQ ID NO: 48 - DNA, 4826 bp, artificial
  MinimalGag-SpCas9 cds
atgggtgcgagagcgtcagtatctagtcctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgctgcagcgtatgaagcagctcgaggataaagtggaagaactcttaagcaaaaactaccacctggaaaacgaagtggcacgtctgaaaaagcttgtgggtgagacagcatccgctcctcctcccccgtacgtaggctctggcggatccggcgggtccggtgccaccgctagcgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtt

SEQ ID NO: 49 - protein, 1939 aa, artificial
  Gag-SpCas9 amino acid sequence
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYRRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYEGRPGNFLQSRPEPTAPPEESFRSGVETTTPPQKQEPIDKELYPLTSLRSLFGSDPSSQGAQGSQNYPIVQTGATMDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 50 - protein, 1587 aa, artificial
  MinimalGag-SpCas9 amino acid sequence
MGARASVSSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGETASAPPPPYVGSGGSGGSGATASDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 51 - DNA, 364 bp, artificial
  pVAX-T7-sgRNA transcriptional unit
ggtacctaatacgactcactataggagacggattgacgtctctgtttaagagctatgctggaaacagcatagcaagtttaaataaggctagtccgttatcaacttgaaaaagtggcaccgagtcggtgcttgggtcggcatggcatctccacctcctcgcggtccgacctgggcatccgaaggaggacgtcgtccactcggatggctaagggagagctcggatccggctgctaacaaagcccgaaaggaagctgagttggctgctgccaccgctgagcaataactagcataaccccttggggcctctaaacgggtcttgaggggttttttgctgaaaggaggaactatatccggatcgagatcc

SEQ ID NO: 52 - DNA, 22 bp, artificial
  gRNA for
taagagctatgctggaaacagc

SEQ ID NO: 53 - DNA, 20 bp, artificial
  gRNA rev
gactcggtgccactttttca

SEQ ID NO: 54 - DNA, 27 bp, artificial
  gRNAopt for
agctagaaatagcaagttaaaataagg

SEQ ID NO: 55 - DNA, 20 bp, artificial
  gRNAopt rev
gactcggtgccactttttca